(19) United States
(12) Patent Application Publication
(10) Pub. No.: US 2014/0346515 A1
(43) Pub. Date: Nov. 27, 2014

(54) SEMICONDUCTOR DEVICE AND METHOD FOR MANUFACTURING SEMICONDUCTOR DEVICE

(71) Applicant: Hitachi, Ltd., Chiyoda-ku, Tokyo (JP)

(72) Inventors: Itaru Yanagi, Tokyo (JP); Masahiko Ando, Tokyo (JP); Toshiyuki Mine, Tokyo (JP); Taro Osabe, Tokyo (JP); Tomoyuki Ishii, Tokyo (JP)

(21) Appl. No.: 14/372,750

(22) PCT Filed: Nov. 19, 2012
(86) PCT No.: PCT/JP2012/079980

§ 371 Date: Jul. 17, 2014

(30) Foreign Application Priority Data
Jan. 18, 2012 (JP) 2012-008289

(51) Int. Cl. H01L 29/786; H01L 29/66

(52) U.S. Cl. CPC H01L 29/78675; H01L 29/66757
USPC 257/48; 257/66; 438/151

(57) ABSTRACT

Detection accuracy of a semiconductor device for detecting various kinds of substances including biological matter such as DNA is to be increased. This semiconductor device includes: a channel region CH placed on a first surface of a silicon oxide film 110; source/drain regions placed on both sides of the channel region CH; a gate electrode G placed on the first surface at a distance from the channel region CH, the gate electrode G being located to face a side surface xz1 of the channel region CH; an insulating film Z located between the channel region CH and the gate electrode G; and a pore P extending parallel to the side surface xz1 of the channel region CH, the pore P being perpendicular to the first surface. A test object such as DNA 200 is introduced into the pore P, and field changes caused by the test object in an inversion layer 10 formed in the side surface xz1 of the channel region CH is detected as changes in the current flowing between the source/drain regions.

### TECHNICAL FIELD

The present invention relates to semiconductor devices, and more particularly, to a technology that is useful in semiconductor devices for detecting various kinds of substances including biological matter such as DNA.

### BACKGROUND ART

All the genomic sequences of many living creatures have been analyzed by using DNA (deoxyribonucleic acid) sequencers. Those genomic sequences are specific to respective individuals, and are the foundation for understanding life phenomena. Therefore, genomic sequence analysis is essential for the development of biology and medicine.

However, genomic sequence analysis takes enormous amounts of time and cost in the current situation, and there is a demand for methods and devices for fast and accurate analysis at low costs.

For example, NPL 1 listed below discloses a nanopore device as a DNA sequencer of a more advanced generation, which includes a pore (a hole) of a size similar to the size of DNA and electrodes on both sides of the pore. NPL 2 listed below discloses the use of a semiconductor process in manufacturing a nanopore device. According to this literature, a region of a thin insulating film is formed on a semiconductor substrate, and two electrodes are formed in the region. A minute pore (a hole) is then formed between the two electrodes by using an electron beam or the like.

PTL 1 listed below discloses a device in which a pore for guiding DNA through the inside thereof is formed in the channel connecting the source and the drain (FIGS. 5a through 5e).

PTL 2 listed below also discloses a device in which a pore for guiding DNA through the inside thereof is formed in the channel connecting the source and the drain. This literature further discloses a structure in which the pore is located at an edge of the channel or outside the channel (FIG. 1 and FIGS. 2A through 2C). NPL 3 listed below discloses an electron density distribution of Si in an insulating film.

### CITATION LIST

### Patent Literatures

PTL 1: US 2011/0133255 A1
PTL 2: US 2010/0327847 A1

### Non-Patent Literatures

NPL 1: Johan Lagerqvist, et al., NANO LETTERS (2006) Vol. 6, No. 4 779-782
NPL 2: Brian C. Gierhart, et al., SENSORS AND ACTUATORS B 132 (2008) 593-600
NPL 3: Bogdan Majkusiak, et al., IEEE TRANSACTION ON ELECTRON DEVICES, VOL 45, NO. 5, MAY 1998

### SUMMARY OF INVENTION

### Technical Problem

In the devices disclosed in NPLs 1 and 2 listed above, however, changes in tunneling current are detected. Therefore, a pair of electrodes with a gap (approximately 1.25 nm, for example) as narrow as the thickness of DNA (approximately 1 nm, for example) need to be prepared. In view of this, it is difficult to manufacture a device with high precision and high reproducibility, even if a semiconductor technology for enabling fine processing is used.

On the other hand, in the device of PTL 1 listed above, which has the DNA passing pore in the channel connecting the source and the drain (FIGS. 5a through 5e), and in the device of PTL 2 listed above, which has the DNA passing pore in the channel connecting the source and the drain (FIG. 1 and FIGS. 2A through 2C), changes in channel potential can be detected as changes in source-drain current, for example. Accordingly, there is no need to form a narrow gap (approximately 1.25 nm, for example) between the source and the drain as in NPLs 1 and 2 listed above. This is highly advantageous in terms of manufacturing.

In the device structures of PTLs 1 and 2 listed above, however, the pore is perpendicular to the channel plane, and it is difficult to conduct testing with high sensitivity. Therefore, there is a demand for devices with higher detection accuracy.

The present invention aims to improve characteristics of semiconductor devices. Particularly, the present invention aims to increase detection accuracy of a semiconductor device for detecting various kinds of substances including biological matter such as DNA. The present invention also aims to provide a method of manufacturing a semiconductor device with excellent characteristics.

Other objects as well as the above mentioned objects, and features of the present invention will become apparent in conjunction with the description in this specification and the accompanying drawings.

### Solution to Problem

The following is a brief description of typical modes of the invention disclosed in this application.

A semiconductor device as a typical mode of the invention disclosed in this application includes: a first semiconductor film placed on a first surface of an insulating layer; source/drain regions placed on both sides of the first semiconductor film; a gate electrode that is placed on the first surface at a distance from the first semiconductor film, and is located to face a first side surface of the first semiconductor film; a first insulating film located between the first semiconductor film and the gate electrode; and a hole that extends parallel to the first side surface of the first semiconductor film, and is perpendicular to the first surface.

A semiconductor device as another typical mode of the invention disclosed in this application includes: a first electrode placed on a first surface of an insulating layer; a second electrode that is placed on the first surface at a distance from the first electrode, and is located to face a first side surface of the first electrode; a first insulating film located between the first electrode and the second electrode; a hole that is formed in the first insulating film, extends parallel to the first side surface of the first electrode, and is perpendicular to the first surface; and source/drain electrodes that face a second surface of the insulating layer, and are located on both sides of the first electrode.

A method of manufacturing a semiconductor device as yet another typical mode of the invention disclosed in this application includes: (a) forming a first semiconductor film on a first surface of an insulating layer, and patterning the first semiconductor film to form a first film piece, a second film piece, and a third film piece; (b) forming a second semiconductor film on the first film piece, the second film piece, and the third film piece; (c) thinning the second semiconductor film by oxidizing a surface of the second semiconductor film; (d) forming a semiconductor region from the second semiconductor film connecting the first film piece and the second film piece by patterning the second semiconductor film; and (e) forming a hole in a region located between the semiconductor region and the third film piece, the region including an inside portion of the semiconductor region.

### Advantageous Effects of Invention

The following is a brief description of the effects to be achieved by a typical mode of the invention disclosed in this application.

In a semiconductor device as a typical mode of the invention disclosed in this application, its characteristics can be improved. Particularly, in a semiconductor device for detecting various kinds of substances, its detection characteristics can be improved. Also, according to a method of manufacturing a semiconductor device as a typical mode of the invention disclosed in this application, a semiconductor device having excellent detection characteristics can be manufactured.

### BRIEF DESCRIPTION OF DRAWINGS

FIG. 1 is a schematic perspective view of a semiconductor device of a first embodiment.

FIGS. 2(A) and 2(B) are a perspective view and a cross-sectional view, respectively, showing the structure in the vicinity of the pore in the semiconductor device of the first embodiment.

FIG. 3(A) is a diagram schematically showing the existence probability of monoelectrons in a Si layer inserted in a silicon oxide film (a SiO₂ film), and FIG. 3(B) is a diagram schematically showing the existence probability of two electrons in a Si layer inserted in a silicon oxide film (a SiO₂ film).

FIG. 4 is a perspective diagram for explaining the relationship between the pore portion of the semiconductor device of the first embodiment and the capacitances shown in FIG. 5.

FIG. 5 is a circuit diagram showing the relationship between the pore portion and the capacitances.

FIG. 6 is a graph showing the results of a simulation of electrical characteristics of the semiconductor device according to the first embodiment.

FIG. 7 is a cross-sectional view of relevant components, illustrating a procedure for manufacturing the semiconductor device of the first embodiment.

FIG. 8 is a cross-sectional view of relevant components, illustrating a procedure for manufacturing the semiconductor device of the first embodiment.

FIG. 9 is a plan view of relevant components, illustrating a procedure for manufacturing the semiconductor device of the first embodiment.

FIG. 10 is a cross-sectional view of relevant components, illustrating a procedure for manufacturing the semiconductor device of the first embodiment.

FIG. 11 is a cross-sectional view of relevant components, illustrating a procedure for manufacturing the semiconductor device of the first embodiment.

FIG. 12 is a plan view of relevant components, illustrating a procedure for manufacturing the semiconductor device of the first embodiment.

FIG. 13 is a cross-sectional view of relevant components, illustrating a procedure for manufacturing the semiconductor device of the first embodiment.

FIG. 14 is a cross-sectional view of relevant components, illustrating a procedure for manufacturing the semiconductor device of the first embodiment.

FIG. 15 is a plan view of relevant components, illustrating a procedure for manufacturing the semiconductor device of the first embodiment.

FIG. 16 is a cross-sectional view of relevant components, illustrating a procedure for manufacturing the semiconductor device of the first embodiment.

FIG. 17 is a cross-sectional view of relevant components, illustrating a procedure for manufacturing the semiconductor device of the first embodiment.

FIG. 18 is a plan view of relevant components, illustrating a procedure for manufacturing the semiconductor device of the first embodiment.

FIG. 19 is a cross-sectional view of relevant components, illustrating a procedure for manufacturing the semiconductor device of the first embodiment.

FIG. 20 is a cross-sectional view of relevant components, illustrating a procedure for manufacturing the semiconductor device of the first embodiment.

FIG. 21 is a plan view of relevant components, illustrating a procedure for manufacturing the semiconductor device of the first embodiment.

FIG. 22 is a cross-sectional view of relevant components, illustrating a procedure for manufacturing the semiconductor device of the first embodiment.

FIG. 23 is a cross-sectional view of relevant components, illustrating a procedure for manufacturing the semiconductor device of the first embodiment.

FIG. 24 is a plan view of relevant components, illustrating a procedure for manufacturing the semiconductor device of the first embodiment.

FIG. 25 is a cross-sectional view of relevant components, illustrating a procedure for manufacturing the semiconductor device of the first embodiment.

FIG. 26 is a cross-sectional view of relevant components, illustrating a procedure for manufacturing the semiconductor device of the first embodiment.

FIG. 27 is a plan view of relevant components, illustrating a procedure for manufacturing the semiconductor device of the first embodiment.

FIG. 28 is a cross-sectional view of relevant components, illustrating a procedure for manufacturing the semiconductor device of the first embodiment.

FIG. 29 is a cross-sectional view of relevant components, illustrating a procedure for manufacturing the semiconductor device of the first embodiment.

FIG. 30 is a plan view of relevant components, illustrating a procedure for manufacturing the semiconductor device of the first embodiment.

FIG. 31 is a cross-sectional view of relevant components, illustrating a procedure for manufacturing the semiconductor device of the first embodiment.

FIG. 32 is a cross-sectional view of relevant components, illustrating a procedure for manufacturing the semiconductor device of the first embodiment.

FIG. 33 is a plan view of relevant components, illustrating a procedure for manufacturing the semiconductor device of the first embodiment.

FIG. 34 is a cross-sectional view of relevant components, illustrating a procedure for manufacturing the semiconductor device of the first embodiment.

FIG. 35 is a cross-sectional view of relevant components, illustrating a procedure for manufacturing the semiconductor device of the first embodiment.

FIG. 36 is a plan view of relevant components, illustrating a procedure for manufacturing the semiconductor device of the first embodiment.

FIG. 37 is a cross-sectional view of relevant components, illustrating a procedure for manufacturing the semiconductor device of the first embodiment.

FIG. 38 is a cross-sectional view of relevant components, illustrating a procedure for manufacturing the semiconductor device of the first embodiment.

FIG. 39 is a plan view of relevant components, illustrating a procedure for manufacturing the semiconductor device of the first embodiment.

FIGS. 40(A) and 40(B) are a perspective view and a plan view, respectively, showing a preferred region in which the pore P is to be located.

FIGS. 41(A) and 41(B) are a perspective view and a plan view, respectively, showing a structure according to Modification 1 of a semiconductor device of a second embodiment.

FIGS. 42(A) and 42(B) are a perspective view and a plan view, respectively, showing a structure according to Modification 2 of the semiconductor device of the second embodiment.

FIGS. 43(A) and 43(B) are a perspective view and a plan view, respectively, showing a structure according to Modification A of a semiconductor device of a third embodiment.

FIGS. 44(A) and 44(B) are a perspective view and a plan view, respectively, showing a structure according to Modification B of the semiconductor device of the third embodiment.

FIGS. 45(A) and 45(B) are a perspective view and a plan view, respectively, showing a structure according to Modification C of the semiconductor device of the third embodiment.

FIGS. 46(A) and 46(B) are a perspective view and a plan view, respectively, showing a structure according to Modification D of the semiconductor device of the third embodiment.

FIGS. 47(A) and 47(B) are a perspective view and a plan view, respectively, showing a structure according to Modification E of the semiconductor device of the third embodiment.

FIG. 48 is a cross-sectional view of relevant components, illustrating the structure of a semiconductor device of a fourth embodiment.

FIG. 49 is a cross-sectional view of relevant components, illustrating the structure of the semiconductor device of a fourth embodiment.

FIG. 50 is a plan view of relevant components, illustrating the structure of the semiconductor device of the fourth embodiment.

FIG. 51 is a cross-sectional view of relevant components, illustrating a procedure for manufacturing the semiconductor device of the fourth embodiment.

FIG. 52 is a cross-sectional view of relevant components, illustrating a procedure for manufacturing the semiconductor device of the fourth embodiment.

FIG. 53 is a cross-sectional view of relevant components, illustrating a procedure for manufacturing the semiconductor device of the fourth embodiment.

FIG. 54 is a cross-sectional view of relevant components, illustrating a procedure for manufacturing the semiconductor device of the fourth embodiment.

FIG. 55 is a schematic cross-sectional view of the structure of a semiconductor device of a fifth embodiment.

FIG. 56 is a schematic cross-sectional view of the structure of another semiconductor device of the fifth embodiment.

FIG. 57 is a schematic perspective view of a semiconductor device of a sixth embodiment.

FIGS. 58(A) and 58(B) are a schematic cross-sectional view and a schematic plan view, respectively, showing the structure in the vicinity of the pore in a semiconductor device of a seventh embodiment.

FIG. 59 is a block diagram schematically showing the configuration of a system according to an eighth embodiment.

FIG. 60 is a block diagram schematically showing the configuration of a system according to the eighth embodiment.

FIG. 61 is a block diagram schematically showing the configuration of a system according to the eighth embodiment.

FIG. 62 is a block diagram schematically showing the configuration of a system according to the eighth embodiment.

FIG. 63 is a block diagram schematically showing the configuration of a system according to the eighth embodiment.

### DESCRIPTION OF EMBODIMENTS

The following is a detailed description of embodiments of the present invention, with reference to the drawings. In all the drawings for explaining the embodiments, components having the same functions or related components are denoted by the same or similar reference numerals, and explanation of them will not be repeated more than once. In the description of the embodiments, explanation of the same or similar aspects will not be repeated, unless necessary. Further, in the drawings for explaining the embodiments, there are shaded portions in some plan views, and there are no shaded portions in some cross-sectional views, for ease of comprehension of the structures. In cross-sectional views and plan views, the sizes of respective components are not necessarily correspond to those of actual devices, and there are cases where some particular components are enlarged relative to the other components, so as to make the drawings clearer. Also, there are cases where the sizes and the positions of respective components might vary among a perspective view, a plan view, and a cross-sectional view that correspond to one another.

### First Embodiment

Referring to drawings, the structure of a semiconductor device of this embodiment and a method of manufacturing the semiconductor device will be described below in detail.

[Description of the Structure]

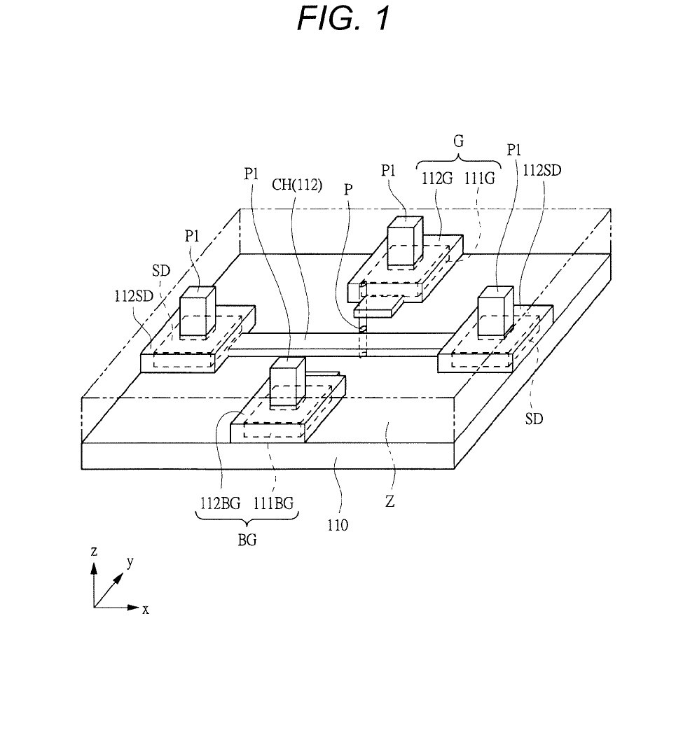

FIG. 1 is a schematic perspective view of the semiconductor device of this embodiment. The semiconductor device of this embodiment is a semiconductor device for bio-related substance detection (a TFT (Thin Film Transistor) for ionic substance detection, a TFT for bio-related substance detection, a TFT for analysis, a semiconductor sensor for analysis and detection, or a biosensor). Here, the bio-related substance is described as DNA, for example.

As shown in FIG. 1, the semiconductor device of this embodiment includes source/drain regions SD, a channel region CH between the source/drain regions SD, and a gate electrode (a control gate electrode) G, which are placed on an insulating film (an insulating layer) such as a silicon oxide film 110. An insulating film Z exists between the channel region CH and the gate electrode G. Thus, the semiconductor device of this embodiment has the structure of a FET (Field Effect Transistor).

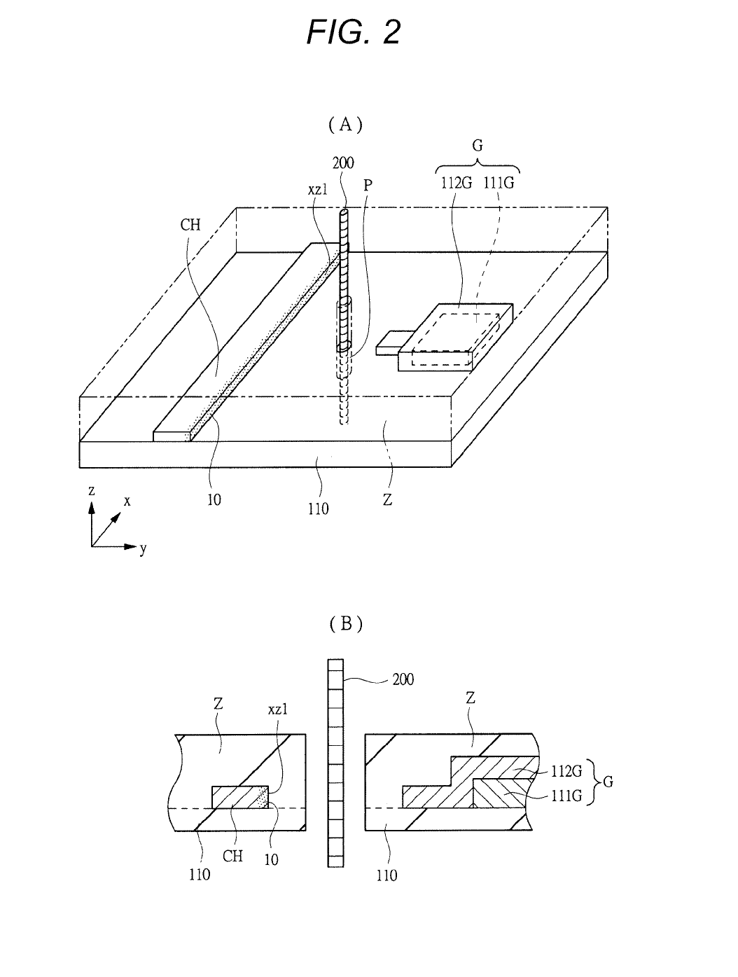

The channel region CH has the shape of a rectangular parallelepiped that has its long side in the x-direction. The source/drain regions SD are placed at both ends of the channel region CH in the x-direction. The source/drain regions SD are the regions to be the source and the drain, and any one of the region may serve as the source (or the drain). The gate electrode G is placed at a predetermined distance on the side of a long-side surface of the channel region CH. The gate electrode G is located on the side of a long-side surface of the channel region CH, and is placed to face a side surface xz1 (a first side surface) that is perpendicular to the upper surface (a first surface) of the silicon oxide film 110 (see FIGS. 2(A) and 2(B)). Part of the insulating film Z exists between the side surface xz1 of the channel region CH and the gate electrode G, and serves as a gate insulating film. Although the insulating film Z is shown as a single-layer film in FIG. 1, this film may be formed with a film stack of insulating films as described later.

Further, a back gate electrode BG is placed on the opposite side of the channel region CH from the gate electrode G. In other words, the back gate electrode BG is placed to face the side surface of the channel region CH on the opposite side from the above mentioned side surface xz1. The back gate electrode BG is not essential in the later described FET operations, and may be removed. However, operations with high controllability can be performed by driving the semiconductor device (FET) with both the gate electrode G and the back gate electrode BG.

The channel region CH is formed with a semiconductor film 112, and this semiconductor film 112 is also placed on the source/drain regions SD. A channel is formed in the side surface xz1 of the semiconductor film 112 (see FIGS. 2(A) and 2(B)). So as to increase influence of a test object (a measurement object or an analysis object) on this channel, it is preferable to minimize the height of the side surface xz1 (the thickness of the semiconductor film 112 or the z-direction thickness). The thickness of the semiconductor film 112 is preferably 5 nm or smaller. If the thickness is 5 nm or smaller, a test object can be examined with high sensitivity. The semiconductor film 112 forming the channel region CH may be an undoped silicon film, for example. Alternatively, a p-type silicon film or a low-density n-type silicon film may be used.

The source/drain regions SD are formed with n-type semiconductor films 111. Here, the semiconductor film (112) is placed on the source/drain regions SD. The semiconductor film is denoted by 112SD.

The gate electrode G is formed with a film stack of an n-type semiconductor film 111 and a semiconductor film 112. Here, the semiconductor films constituting the gate electrode G are denoted by 111G and 112G. The semiconductor film 112G is thinner than the semiconductor film 111G. The semiconductor film 112G covers the upper portion of the semiconductor film 111G and the side surface of the semiconductor film 111G facing the channel region CH, and extends onto the upper portion of the silicon oxide film 110. In other words, the semiconductor film 112G extends from the upper layer of the semiconductor film 111G to the upper surface of the silicon oxide film 110.

As the gate electrode G is formed as a stack structure as described above, the thickness of the side facing the side surface xz1 of the channel region CH can be made smaller, and influence of a gate potential on the channel region CH, particularly on the side surface xz1, can be made greater. Accordingly, a test object can be examined with high sensitivity.

The back gate electrode BG is formed with a film stack of an n-type semiconductor film 111 and a semiconductor film 112. Here, the semiconductor films constituting the back gate electrode BG are denoted by 111BG and 112BG. The semiconductor film 112BG is thinner than the semiconductor film 111BG. The semiconductor film 112BG covers the upper portion of the semiconductor film 111BG and the side surface of the semiconductor film 111BG facing the channel region CH, and extends onto the upper surface of the silicon oxide film 110.

First plugs P1 are placed on the source/drain regions SD, the gate electrode G (111G), and the back gate electrode BG (111BG).

Potentials are applied to the gate electrode G and the back gate electrode BG via the first plugs P1. Predetermined potentials can be applied to the source/drain regions SD via the first plugs P1. Also, a current between the source/drain regions SD can be detected via the first plugs P1 by using an ammeter or the like.

A pore (a hole, a through hole, or an opening) P that penetrates through the insulating film Z and the silicon oxide film 110 is formed in the region between the side surface xz1 of the channel region CH and the gate electrode G. The pore P is a hole (an opening) through which a test object such as a bio-related substance like DNA passes. The diameter of the pore P may be adjusted in accordance with the size of the test object, and is preferably not smaller than 1 nm and not greater than 5 nm when DNA passes therethrough, for example. Since the thickness of DNA is approximately 1 nm, the diameter of the pore P is preferably not smaller than 1 nm, and is preferably not greater than 5 nm so as to examine the test object with high sensitivity.

[Description of Operations]

FIGS. 2(A) and 2(B) are a perspective view and a cross-sectional view of the structure in the vicinity of the pore in the semiconductor device of this embodiment. As shown in the drawings, DNA 200 passes through the inside of the pore P. DNA has a structure in which four types of nucleotides (dAMP, dCMP, dGMP, and dTMP) are aligned. A nucleotide is a substance formed with a phosphate group linked to a nucleoside. A nucleoside is formed by glycosidic linkage of a purine base or a pyrimidine base to the 1 position of a pentose. Of each of the above four-letter abbreviations, the first letter indicates the type of sugar (ribonucleotide (r) or deoxyribonucleotide (d)), the second letter indicates the type of base, the third letter indicates the number of linked phosphate groups (mono: 1, di: 2, tri: 3), and the fourth letter indicates a phosphate (P). Of the types of bases, “G” represents guanine (2-amino-6-oxopurine), “A” represents adenine (6-aminopurine), “T” represents thymine (5-methyluracil), and “C” represents cytosine (2-hydroxy-6-aminopyrimidine).

Respective blocks (pieces) of the DNA 200 represent respective nucleotides, and each of the blocks corresponds to one of the above described types: dAMP, dCMP, dGMP, and dTMP.

When the semiconductor device of this embodiment is made to perform a FET operation, predetermined potentials are applied to the source/drain regions SD, for example (see FIG. 1). Specifically, a first potential (a ground potential, for example) is applied to one of the source/drain regions SD, and a second potential (a power supply potential, for example) that is higher than the first potential is applied to the other one of the source/drain regions SD. By controlling the voltage of the gate electrode G in this situation, the semiconductor device of this embodiment can be made to perform a FET operation.

When a potential such as the second potential (the power supply potential) that is higher than the first potential is applied to the gate electrode G, an inversion layer (a channel) 10 is formed in the side surface xz1 of the channel region CH facing the gate electrode G, as shown in FIGS. 2(A) and 2(B). Accordingly, current flows between the source/drain regions SD via the inversion layer 10. The width of the inversion layer 10 in the y-direction can be adjusted in the range of approximately 1 nm to 10 nm in accordance with the voltages and the like to be applied to the gate electrode and the back gate electrode.

The current (channel current) between the source/drain regions SD varies depending on the four types of nucleotides that pass through the pore P. This is because the nucleotides have different effective charge amounts and different effective electric fields from one type to another. Therefore, in a case where the amperages corresponding to dAMP, dCMP, dGMP, and dTMP are determined to be A1, A2, A3, and A4, respectively, by a test or a simulation using existing DNA, unknown DNA is measured with the semiconductor device (FET) of this embodiment. When the current between the source/drain regions SD varies from A4 to A3 to A1 to A2 to A1 to A4, . . . , it is clear that the nucleotides are aligned in the following order: dTMP, dGMP, dAMP, dCMP, dAMP, dTMP, . . . .

For example, the pitch of the base moiety sequence in the respective nucleotides constituting DNA is approximately 0.34 nm. As described above, the area of the side surface xz1 of the channel region CH can be reduced by forming the channel region CH (the semiconductor film 112) with a thin film of 5 nm or less in thickness. As a result, the effective charge amount (the effective electric field) of one base with respect to the channel area can be made larger. In this manner, a field change caused by one base can be efficiently detected by the semiconductor device of this embodiment.

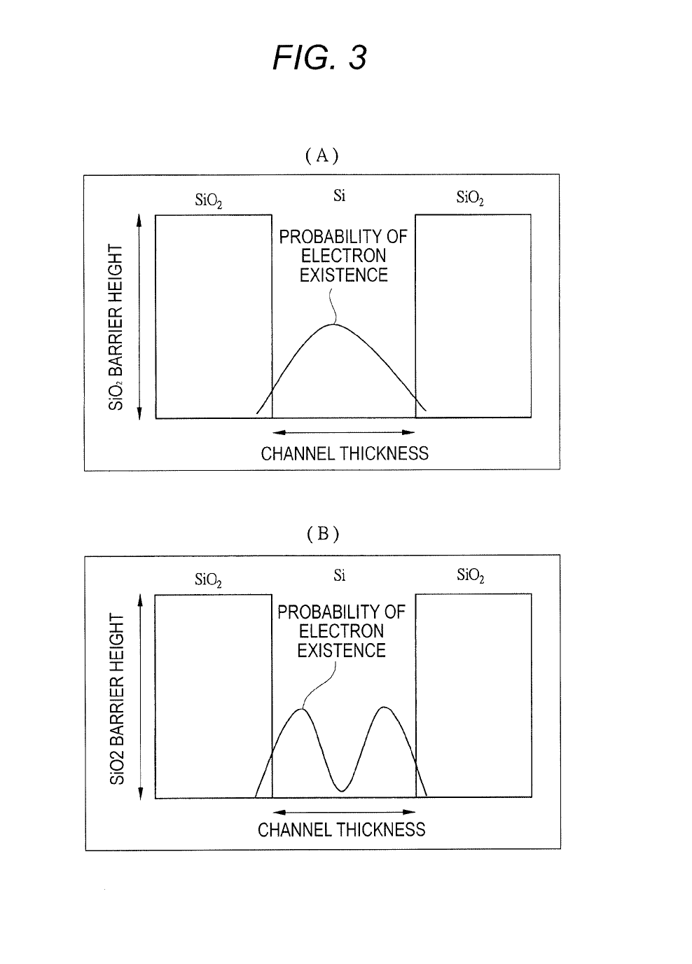

As for the electron density of a Si layer in a silicon oxide film (a SiO₂ film), for example, the electron density distribution (the electron existence probability) concentrates on the center of the layer in a case where the thickness of the Si layer (the thickness of the channel) is nm or smaller (see NPL 3, which has been described above). Further, in a case where the thickness of the Si layer (the thickness of the channel) is 5 nm or smaller, the peak value of the electron density distribution (the electron existence probability) becomes higher, and the graph becomes sharper. In a case where the thickness of the Si layer (the thickness of the channel) is 5 nm or smaller, it is unlikely that two or more electrons exist next to one another in the channel. FIG. 3(A) schematically shows the existence probability of monoelectrons in a Si layer inserted in a silicon oxide film (a SiO₂ film). FIG. 3(B) schematically shows the existence probability of two electrons in a Si layer inserted in a silicon oxide film (a SiO₂ film). The ordinate axis indicates SiO₂ barrier height.

In a case where the Si layer becomes thinner, the system (status) shown in FIG. 3(B) is in a larger energy state (unstable state) than the system (status) shown in FIG. 3(A). As the Si layer becomes thinner, the “difference” between the energy of the system shown in FIG. 3(A) and the energy of the system shown in FIG. 3(B) becomes equal to or larger than room temperature energy (K_{B}T) In such a case, electrons supplied from the source region are mostly in the one-electron state shown in FIG. 3(A) in the thickness direction of the Si layer (the channel) even at room temperature.

As described above, the width (the z-direction width) of the inversion layer (the channel) formed in the side surface xz1 can be reduced by thinning the channel region CH. Particularly, by reducing the thickness of the channel region CH to 5 nm or smaller, a quantum well confinement effect occurs, and a quasi-one-dimensional thin current path in which monoelectrons are aligned in the x-direction can be formed. As such a quasi-one-dimensional thinnest current path is formed as the inversion layer 10, a minute field change due to a test object (bases in the respective nucleotides constituting DNA in this case) in the pore P can be detected with high sensitivity. Accordingly, the rate of change in detection signals (detection sensitivity) can be made higher. Also, with the quasi-one-dimensional thin current path in which monoelectrons are aligned in the x-direction, sufficient spatial resolution is achieved to detect signals of respective bases of a DNA chain aligned with the pitch of approximately 0.34 nm.

Also, an increase in the width of the inversion layer in the y-direction can be restrained by applying a potential that is complementary to the gate electrode G, such as the first potential (the ground potential) or a negative potential, to the back gate electrode BG. In this manner, a stable quasi-one-dimensional current path can be formed by using the back gate electrode BG.

[Results of Operation Simulations]

An example of an operation to analyze the nucleotide sequence of DNA in the above described semiconductor device (see FIG. 1 and FIGS. 2(A) and 2(B)) is described below based on a simulation.

First, differences among effective electric fields generated by respective nucleotides and apparent charge numbers were calculated from the polarizations of the four types of nucleotides (dAMP, dCMP, dGMP, and dTMP). The calculations of the polarizations (dipole moments or quadrupole moments) of the four types of nucleotides were performed by a hybrid DFT method using Gaussian98, which is molecular calculation software of Gaussian, Inc. As a result of calculations of electric fields based on those values, it was confirmed that there were differences among the electric fields generated in the surrounding areas by the four types of nucleotides (bases). For example, at a location 1.5 nm away from the site (the site of the pore P) where the respective nucleotides exist, the electric field generated by a nucleotide toward the polarization in the silicon oxide film (the SiO₂ film) is 2.268 MV/cm in the case of dAMP, is 2.373 MV/cm in the case of dCMP, is 1.952 MV/cm in the case of dGMP, and is 2.163 MV/cm in the case of dTMP. From this result, it also became apparent that there are sufficiently detectable differences among the effective electric fields generated by the respective nucleotides. Meanwhile, the electric field generated by a point charge is 1.537 MV/cm at a location 1.5 nm away from the site (the site of the pore P) where the point charge exists. Where the effective charge number (the apparent charge number) of a test object is defined as the number obtained by dividing the electric field generated by the test object by the electric field generated by the point charge, the apparent charge number of a nucleotide is 1.47 in the case of dAMP, is 1.543 in the case of dCMP, is 1.269 in the case of dGMP, and 1.406 in the case of dTMP.

At a time of testing (driving), an electric field is generated between the gate electrode G and the inversion layer (the channel region CH). In this case, there is a high possibility that the direction of the polarization of each nucleotide is parallel to the direction of the electric field. This is because it becomes stable in terms of energy when the direction of polarization is parallel to the direction of electric field. Therefore, it is considered that electric field modulation based on the effective electric field and the apparent charge number is caused by each nucleotide in the vicinity of the pore P in the inversion layer 10 in a case where the distance between the pore P and the channel region CH is set at approximately 1.5 nm. This electric field modulation can be certainly detected as a difference in current between the source/drain regions SD, for example.

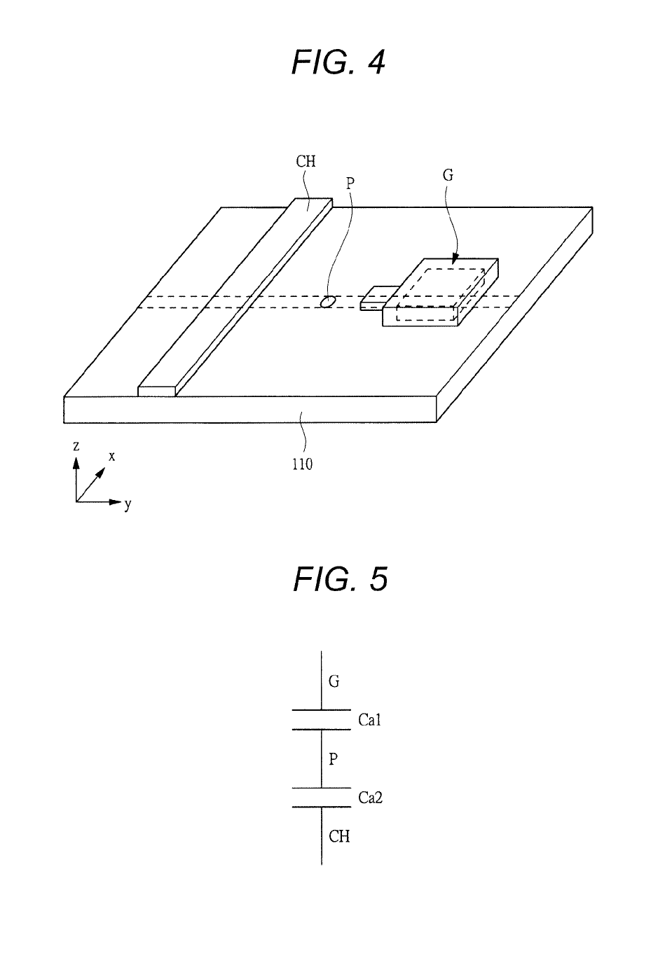

The semiconductor device of this embodiment can be modeled with two capacitances (Ca1 and Ca2) (FIG. 5) sandwiching the pore P in the region between the dashed lines shown in FIG. 4. FIG. 4 is a perspective diagram for explaining the relationship between the pore portion of the semiconductor device of this embodiment and the capacitances shown in FIG. 5. FIG. 5 is a circuit diagram showing the relationship between the pore portion and the capacitances. The capacitance Ca1 shown in FIG. 5 indicates the capacitance between the gate electrode G and the pore P in the region between the dashed lines shown in FIG. 4. The capacitance Ca2 indicates the capacitance between the pore P and the channel region CH in the region between the dashed lines shown in FIG. 4.

For example, the pore P has a shape similar to a 3-nm square prism, the distance between the gate electrode G and the pore P is 50 nm, the thickness of the channel region CH and the thickness of the end portion (the semiconductor film 112G) of the gate electrode G are 3 nm, and the insulating film Z located between the gate electrode G and the pore P is a silicon oxide film (with a relative permittivity of 3.9). In this case, the capacitance Ca1 is approximately 6.21×10⁻²¹ F.

Where a change in charge amount in the pore is ΔQ, the threshold shift amount ΔVth at the edge of the inversion layer (the channel) near the pore is expressed as

ΔVth=ΔQ/Ca1 (Equation 1).

If there is a change of a monoelectron as ΔQ, for example, ΔVth is 25.75 V. In this manner, a very large change in threshold value (Vth, threshold potential, or threshold voltage) is obtained.

As described above, in this embodiment, a quasi-one-dimensional thin current path in which monoelectrons are aligned in the x-direction can be formed by reducing the thickness of the channel region CH. Accordingly, a field change at the edges of the inversion layer (the channel) 10 near the pore P is substantially reflected by the threshold (Vth) shift of the FET. In a case where the width of the inversion layer 10 is large in the z-direction, and the width of the inversion layer 10 is also large in the y-direction, a large threshold shift is not obtained even if a single charge comes close. This is because the influence of the charge is small at a site far away from the charge, and current flows in the inversion layer via the site.

For example, in a case where the difference between dAMP and dTMP (1.47−1.406=0.064), which is the smallest difference among the above described effective charge numbers (the apparent charge numbers), is assigned to ΔQ in the (Equation 1), ΔVth is 1.648 V, which is apparently large enough to be recognized as a difference in threshold value between those nucleotides.

Also, the threshold shift amount becomes larger as the pore P and the channel region CH are brought closer to each other. This is because, of the electric fields existing between the gate electrode G and the channel region CH, the electric field that extends through the side of the pore P and affects the channel region CH can be lowered. As a result, electric field modulation in the pore P can be efficiently transferred to the channel region CH, and the threshold shift amount can be further increased. For example, the distance between the pore P and the channel region CH is preferably 10 nm or shorter. As will be described later in detail in the second embodiment, the pore P and the channel region CH may be in contact with each other, or the pore P may be formed in the channel region CH.

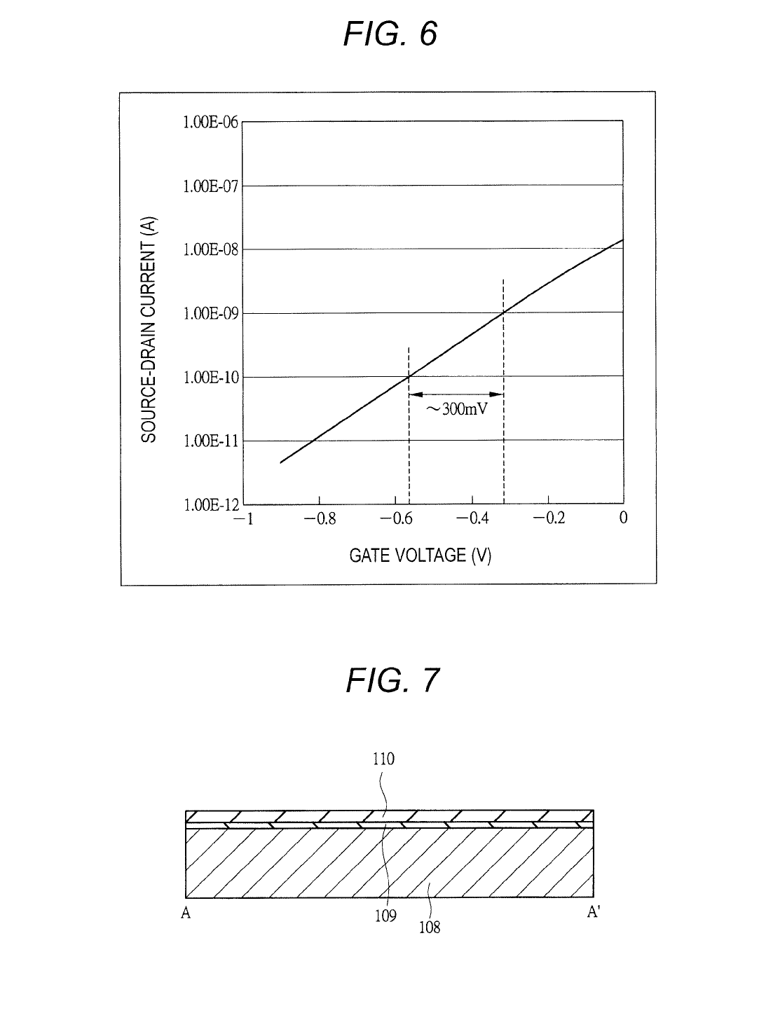

Next, results of a simulation of electrical characteristics of the semiconductor device according to this embodiment are described. FIG. 6 is a graph showing the results of the simulation of the electrical characteristics of the semiconductor device according to this embodiment. The abscissa axis indicates gate voltage (V), and the ordinate axis indicates source-drain current (A). As for the simulation, a simulation of 3D electrical characteristics was performed by using DevEdit and ATLAS, which are device simulators manufactured by SILVACO, Inc. The simulation conditions are as follows. As for the channel region CH, the width (the length in the y-direction) was 50 nm, the length (the length in the x-direction) was 150 nm, and the thickness of the channel region CH (the length in the z-direction) was 3 nm. The gate electrode G, the back gate electrode BG, and the source/drain regions SD were n-type silicon films having a phosphorus (P) density of 3×10²⁰/cm³, and the channel region CH was an undoped silicon film. The distance between the gate electrode G and the channel region CH was 50 nm, and the distance between the channel region CH and the back gate electrode BG was 50 nm. The potential of one (the source side) of the source/drain regions SD was 0 V, the potential of the other one (the drain side) was 0.1 V, the potential of the back gate electrode BG was −2 V, and the potential of the gate electrode G was swept from −1 V to 0 V. It should be noted that 1.00E-n in FIG. 6 indicates 1.00×10⁻ⁿ.

As a result of the above simulation, it was confirmed that the current flowing between the source/drain regions SD concentrated particularly on the edge portions of the channel region CH facing the gate electrode G. It was also confirmed that the source-drain current became higher with an increase in gate voltage, and the S value was approximately 300 mV in the FET characteristics, as shown in FIG. 6. The S value is the gate voltage required to increase the source-drain current by 10-fold. As the S value becomes smaller, the change in the source-drain current becomes larger, and detection sensitivity becomes higher.

For example, in a case where the S value is 300 mV, if a threshold shift ΔVth of 1.648 V based on a difference in effective charge number between dAMP and dTMP is caused, a 5-digit (10⁵) or more current difference can be detected theoretically. In this manner, high-sensitivity DNA analysis can be conducted.

As described above, the semiconductor device of this embodiment is capable of detecting charges of nucleotides as bio-related substances and identifying the types of them. However, the numerical values used in the above described simulations are merely examples, and numerical values that can be used are not limited to those values.

For example, in a case where the change in the source-drain current does not need to be a 5-digit (10⁵) value or greater, the change in the current may be adjusted by increasing the distance between the pore P and the channel region CH. Also, in a case where the change in the source-drain current becomes too small due to a change in the conditions (potentials and impurity densities), the distance between the pore P and the channel region CH should be shortened. As the pore P is provided between the channel region CH and the gate electrode G as described above, the distances between them can be readily adjusted. Accordingly, the change (detection sensitivity) in source-drain current can be readily adjusted. Alternatively, the change in the source-drain current may be adjusted by controlling not the distance therebetween but the permittivity of the insulating film Z located therebetween. For example, the threshold voltage shift amount is increased by using an insulating film having a higher permittivity as the insulating film located between the pore P and the gate electrode G. As a result, the change in the source-drain current can be increased. In this manner, structural design may be adjusted so that detection sensitivity can be increased while usable potentials and withstand voltage are collectively taken into consideration.

Although the above described simulations were performed based on the differences among the effective charges of respective pieces (respective nucleotides) of a test object, detection sensitivity may be increased by performing predetermined processing on the test object solution (the respective nucleotides). For example, the pH of the solution containing the nucleotides is adjusted. In this case, the number of positive or negative ions in the pore P varies with the types of the nucleotides in the pore P. This is due to differences in polarization and size among the nucleotides. Accordingly, the differences in effective electric field and apparent charge number can be made larger by adjusting the pH of the solution and greatly changing the number of ions following the nucleotides.

Also, the test object can be efficiently guided into the pore P by applying a predetermined potential to the test object (the solution containing nucleotides, for example) existing above and below the pore P.

As described above, the back gate electrode BG is not an essential component, but a larger proportion of the inversion layer 10 can be formed on the side of the pore P by applying a predetermined potential to the back gate electrode BG. In other words, the width of the inversion layer 10 in the y-direction can be made smaller. Also, threshold value adjustment can be performed by applying a predetermined potential to the back gate electrode BG (see FIGS. 2(A) and 2(B)).

In the above described simulations, at a time of testing (driving), the direction of the polarization of each nucleotide is assumed to be parallel to the direction of the electric field. However, there is a certain probability that the direction of polarization is not parallel to the direction of electric field. Accordingly, the accuracy of the test can be increased by guiding the same sample (the above described solution, for example) through the pore P a number of times, and repeatedly performing the measurement.

As described above, the semiconductor device (FET) of this embodiment is capable of greatly increasing detection sensitivity. Accordingly, the semiconductor device can detect a minute change in charge amount of a bio-related substance such as DNA, and conduct genomic analyses and the like, which normally cost enormous amounts of time and cost, with efficiency and precision at low costs. Accordingly, the semiconductor device is suitably used in analyzing a bio-related substance such as DNA. However, objects to be tested by the semiconductor device of this embodiment are not limited to DNA, and the semiconductor device can be applied widely to testing of other bio-related substances and substances from which effective charge amounts and effective electric fields can be detected (substances with charges, for example).

Using a different technique from the tunneling current technique of NPLs 1 and 2 described above, the semiconductor device of this embodiment detects DNA of approximately 1 nm in thickness. Accordingly, the distance between electrodes does not need to be shortened (to approximately 1.25 nm, for example), and testing can be performed with higher precision.

By the tunneling current technique, the end portions of the electrodes are exposed, and therefore, there is a risk of characteristics degradation due to oxidation of the electrodes or the like. In the semiconductor device of this embodiment, on the other hand, the electrodes (SD, G, and BG) are covered with an insulating film, and therefore, degradation of the electrodes can be reduced.

Furthermore, the semiconductor device of this embodiment has higher detection sensitivity than in a case where a pore is formed in the current flow path (channel) or where a pore is formed perpendicularly to the channel plane as disclosed in PTLs 1 and 2 described above. As the channel width (current width) becomes larger relative to the diameter of the pore, the change in current caused by the existence/nonexistence or a change of a test object in the pore becomes smaller. With the structures disclosed in PTLs 1 and 2 described above, it is difficult to set a channel width (current width) that is almost the same as the diameter of the pore (approximately 3 nm, for example). In this embodiment, on the other hand, a thin current path (the inversion layer 10) can be formed in the side surface xz1 of the channel region CH, and detection sensitivity can be dramatically increased (see FIGS. 2(A) and (B), and others).

[Description of a Manufacturing Method]

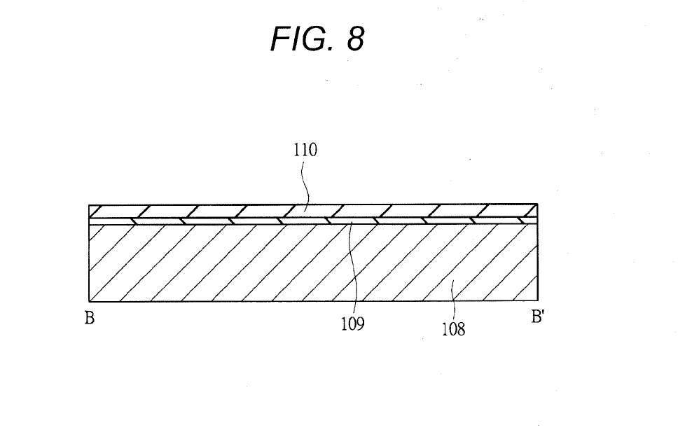

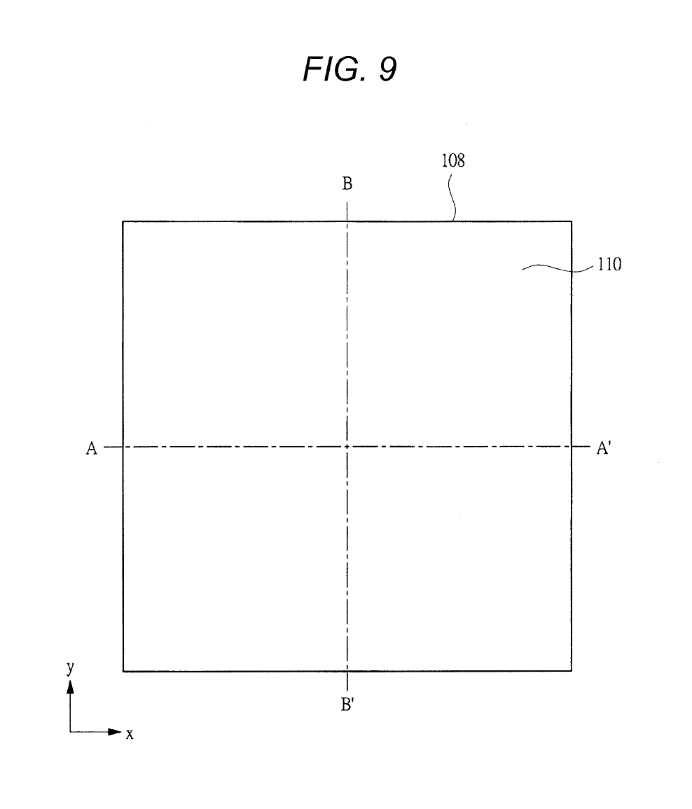

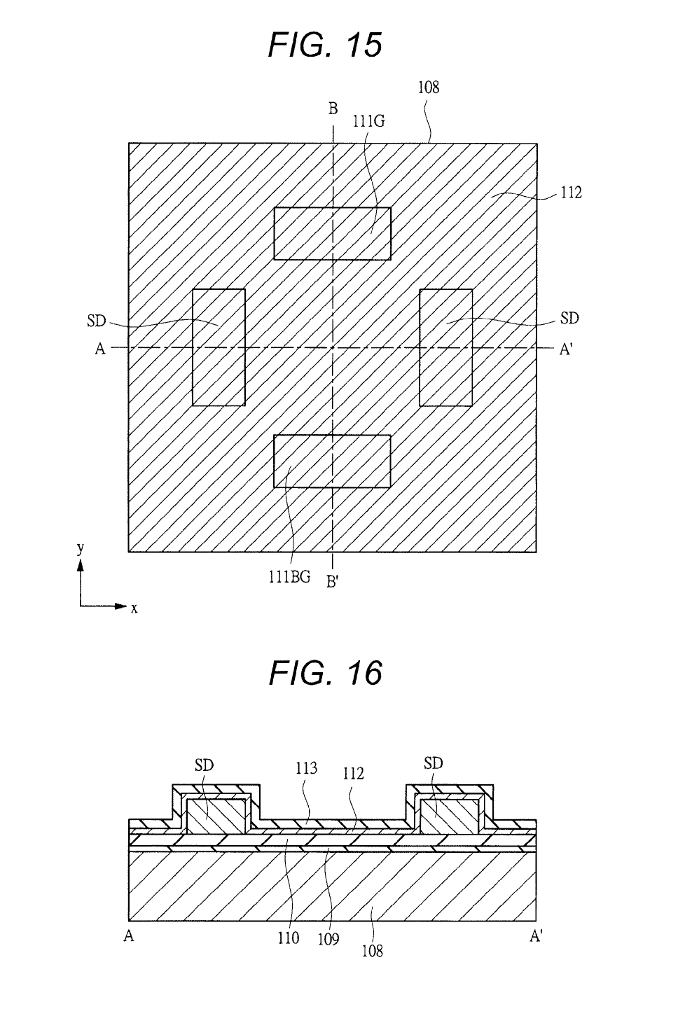

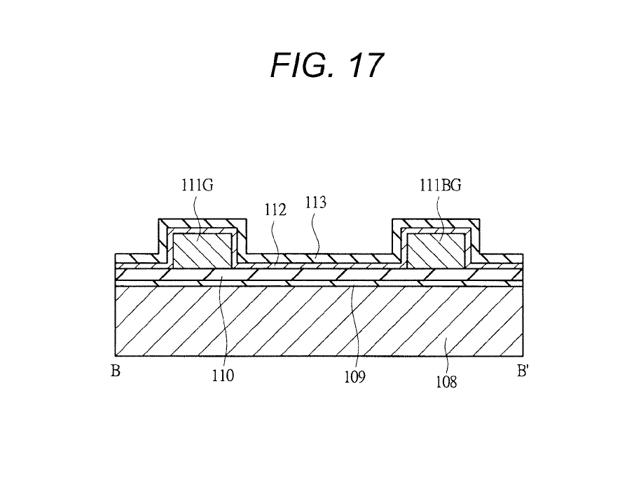

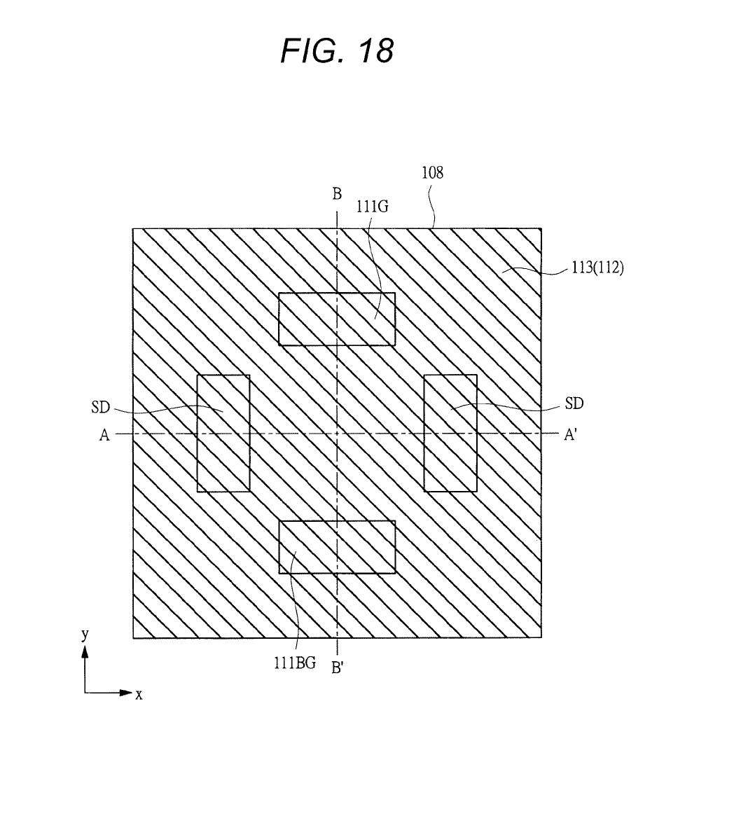

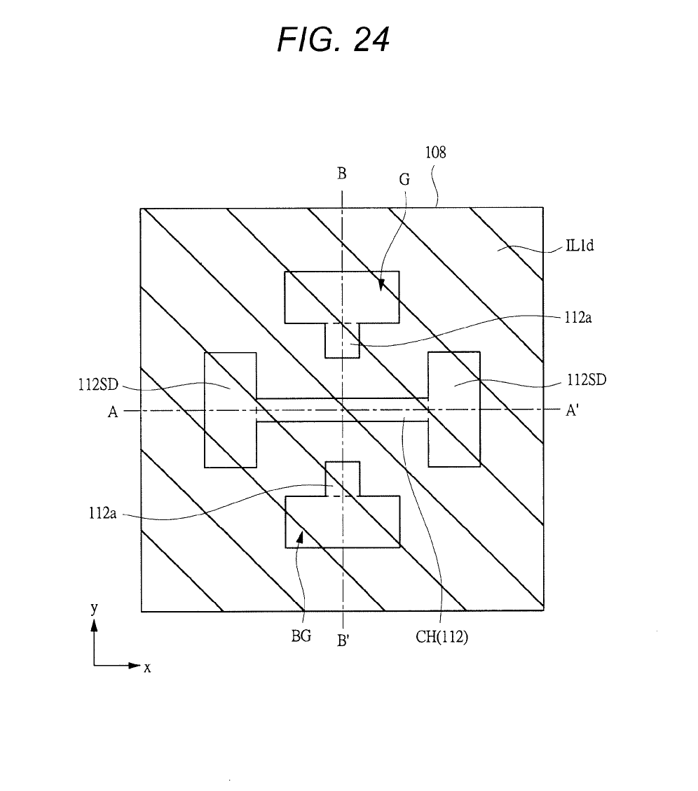

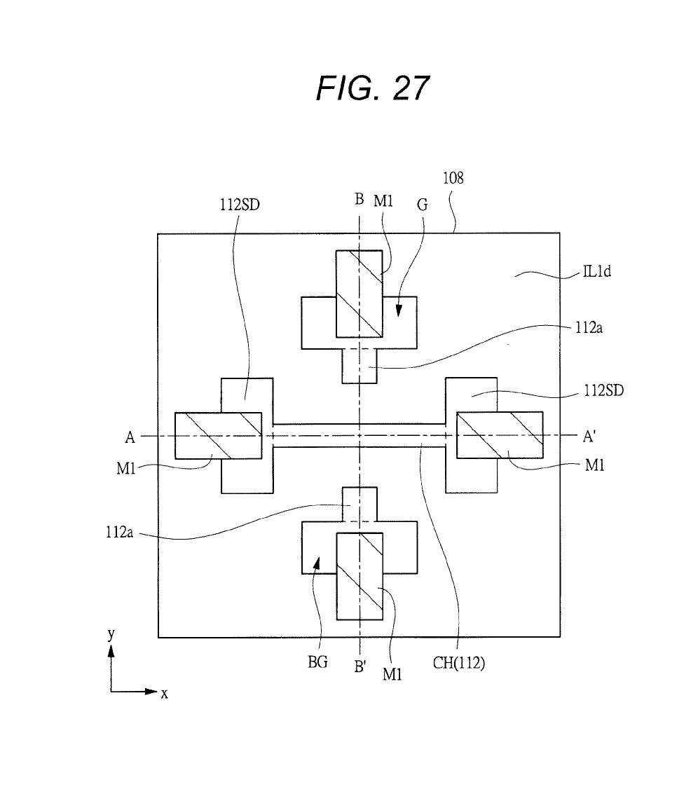

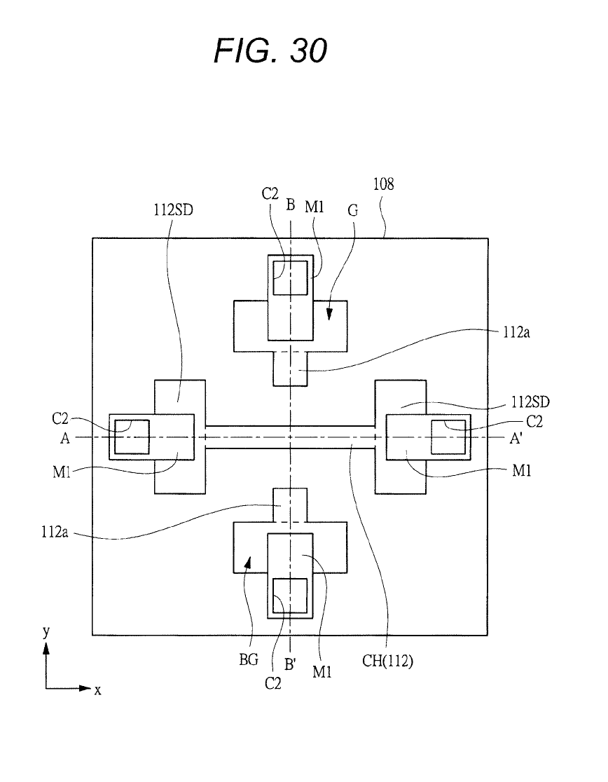

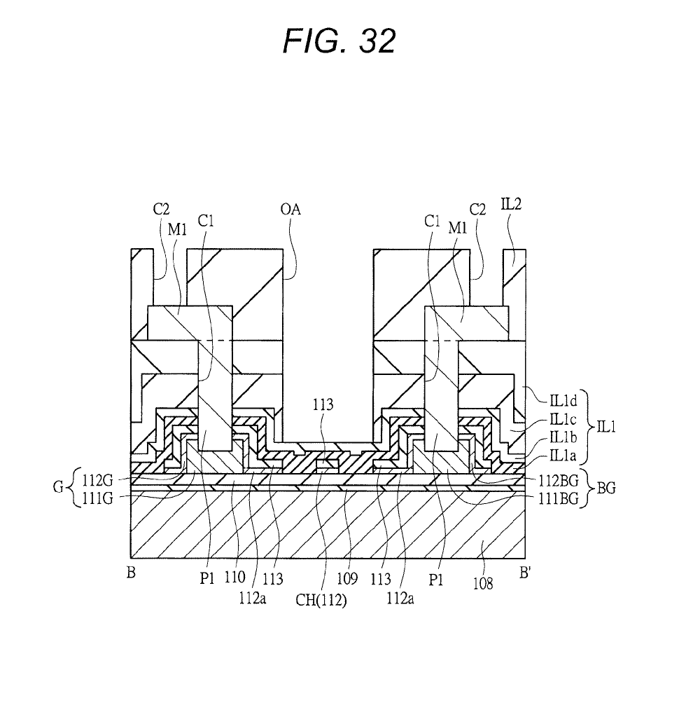

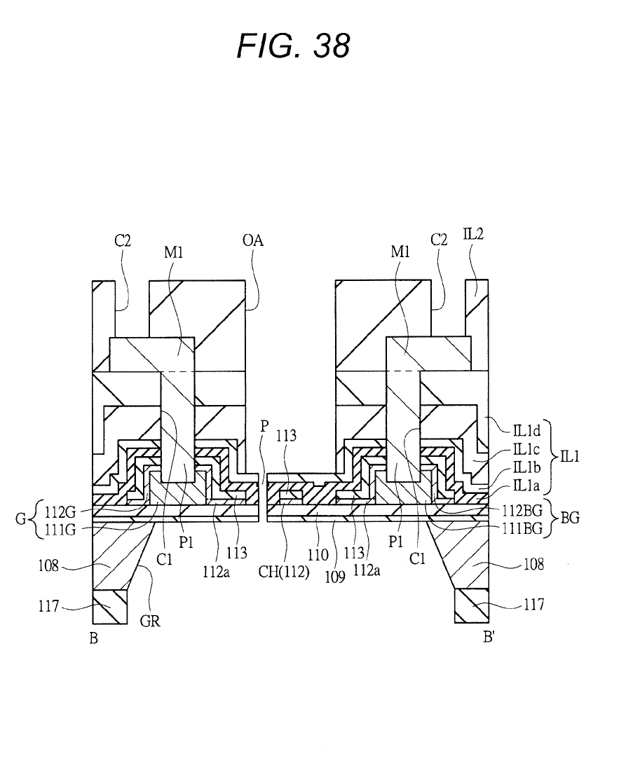

Referring now to FIGS. 7 through 39, a method of manufacturing the semiconductor device of this embodiment is described, and the structure of this semiconductor device is described in greater detail. FIGS. 7 through 39 are cross-sectional views or plan views of relevant components, illustrating the procedures for manufacturing the semiconductor device of this embodiment. The cross-sectional views are equivalent to A-A′ cross-sections or B-B′ cross-sections of the plan views.

First, as shown in FIGS. 7 through 9, a silicon substrate, for example, is prepared as a supporting substrate 108. Here, a substrate other than a silicon substrate may be used. A film stack of a silicon nitride film 109 and the silicon oxide film 110, for example, is formed as an underlayer insulating film on the supporting substrate 108. The silicon nitride film 109 having a thickness of approximately 15 nm, for example, is deposited by using CVD (Chemical Vapor Deposition) or the like. The silicon oxide film 110 having a thickness of approximately 15 to 30 nm, for example, is deposited thereon by CVD or the like.

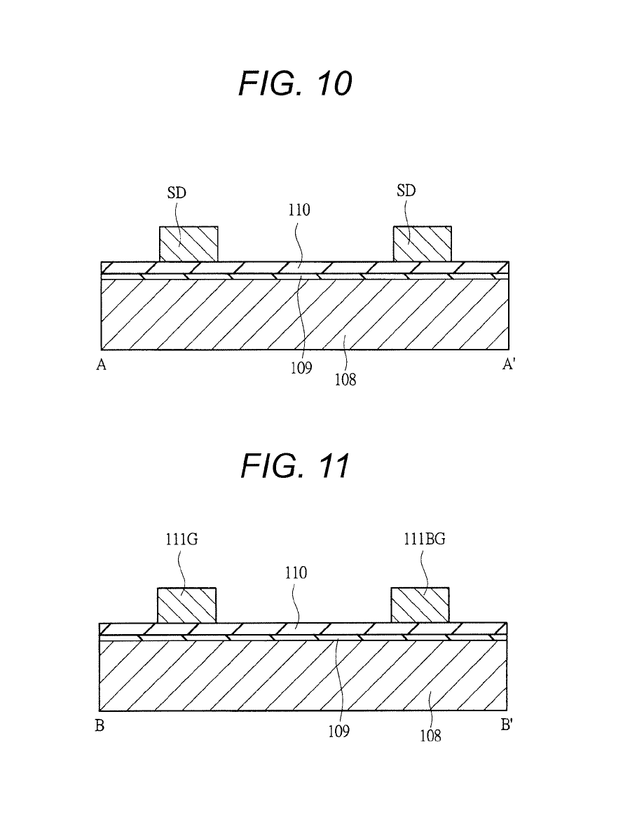

As shown in FIGS. 10 through 12, an n-type polycrystalline silicon film, for example, is then formed as a semiconductor film (a conductive film) on the underlayer insulating film (the silicon oxide film 110). The n-type polycrystalline silicon film having a thickness of approximately 100 nm, for example, is deposited by CVD or the like, while being doped with an n-type impurity, for example.

A photoresist film (not shown) is then formed by a photography technique in the regions in which the source/drain regions SD, the gate electrode G, and the back gate electrode BG are to be formed, and etching is performed on the n-type polycrystalline silicon film (semiconductor film) by using the photoresist film as a mask. After that, the photoresist film is removed by asking or the like, to form the source/drain regions SD, the semiconductor film 111G and the semiconductor film 111BG. The series of procedures from photolithography to the removal of the photoresist film is called patterning. That is, the three patterns (film pieces) to be the source/drain regions SD, the semiconductor film 111G, and the semiconductor film 111BG are formed by patterning a semiconductor film.

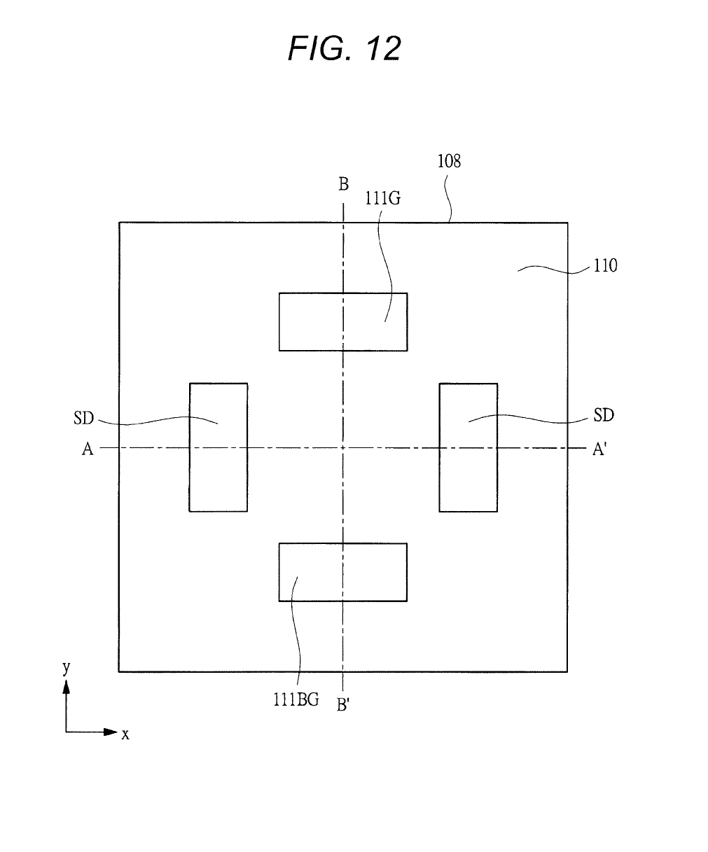

The source/drain regions SD are the regions to be the source and the drain, and any one of the region may serve as the source (or the drain). As shown in FIG. 12, the patterns (the planar shapes seen from above) of the source/drain regions SD, the semiconductor film 111G, and the semiconductor film 111BG are rectangles. The two source/drain regions SD are aligned in the x-direction at a predetermined distance from each other. A region between the two source/drain regions SD is to be the channel region CH described later. The semiconductor film 111G and the semiconductor film 111BG are aligned in the y-direction at a predetermined distance from each other.

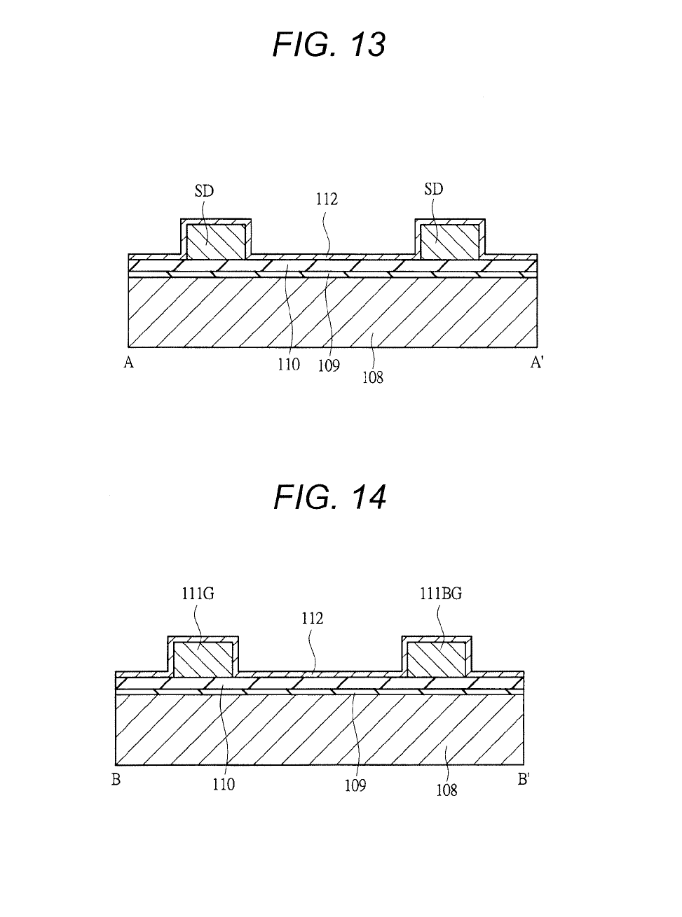

As shown in FIGS. 13 through 15, an undoped polycrystalline silicon film, for example, is then formed as a semiconductor film 112 on the underlayer insulating film (the silicon oxide film 110) as well as on the source/drain regions SD, the semiconductor film 111G, and the semiconductor film 111BG. This semiconductor film 112 is to form the channel region CH. This semiconductor film 112 is also to form the gate electrode G and the back gate electrode BG with the lower semiconductor films (111G and 111BG).

After an amorphous silicon film is deposited by CVD or the like, for example, the amorphous silicon film is polycrystallized by a heat treatment (an annealing treatment), to form a polycrystalline silicon film. Diffusion of the n-type impurity (dopant) into the polycrystalline silicon film is adjusted by appropriately controlling the heat treatment period and temperature. That is, diffusion of the n-type impurity (dopant) into the later described channel region CH and the thin film portions (112G and 112BG) of the gate electrode G and the back gate electrode BG is adjusted. If the impurity diffuses into the entire channel region CH, a FET operation cannot be performed. Therefore, the heat treatment period and temperature are set so that the impurity will not diffuse into the center portion of the channel region CH. The thin film portion (112G or 112BG) of the gate electrode G or the back gate electrode BG preferably has resistance that is as low as possible, so as to serve as the gate electrode (G or BG) applying an electric field to the channel region CH. In view of this, the heat treatment period and temperature are controlled so that the impurity will diffuse into the thin film portion (112G or 112BG) of the gate electrode G or the back gate electrode BG while preventing diffusion of the impurity into the area surrounding the center portion of the channel region CH.

The introduction of the impurity into the thin film portion (112G or 112BG) of the gate electrode G or the back gate electrode BG may be performed by an ion implantation technique. For example, the source/drain regions SD, the semiconductor films 111G, 111BG, 112SD, 112G, and 112BG, and the channel region CH are formed with undoped polycrystalline silicon films, and the impurity is introduced by ion implantation, with a mask, into the channel region CH or the region in which the channel region CH is to be formed. After that, short-time activation annealing is performed to activate the impurity. The impurity activation annealing may be RTA (Rapid Thermal Annealing) or LSA (Laser Spike Annealing), for example. By the procedure using such an ion implantation technique, diffusion of the impurity into the channel region CH can be reduced, and a wider portion can be secured as the portion to function as the channel in the channel region CH.

The thickness of the polycrystalline silicon film to be the channel region is preferably 5 nm or smaller. A thin film of 2 nm or smaller in thickness can be formed as the polycrystalline silicon film. As the thin semiconductor film 112 (the polycrystalline silicon film) is formed in this manner, the channel region (semiconductor region) CH facing the gate electrode G can be made thinner. Accordingly, at a time of driving, a quasi-one-dimensional thin current path (the inversion layer 10) can be formed in the side surface xz1 of the channel region CH (see FIGS. 2(A) and 2(B)).

As shown in FIGS. 16 through 18, an oxidation treatment is performed on the amorphous silicon film or the amorphous silicon film turned into the polycrystalline silicon film after the polycrystallization, for example. Through the oxidation treatment, the surface of the silicon film is oxidized, and a silicon oxide film 113 is formed. In this procedure, the thickness of the semiconductor film (silicon film) 112 remaining under the silicon oxide film 113 can be reduced. If the thickness of the semiconductor film 112 (the amorphous silicon film or the polycrystalline silicon film in this case) can be controlled with high precision, the above described oxidation treatment (oxidation process) may be skipped.

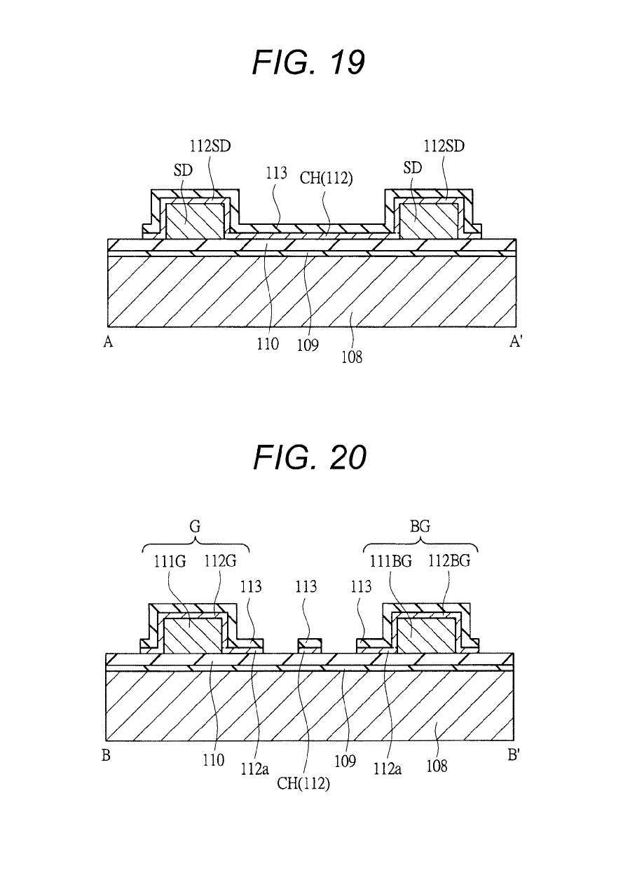

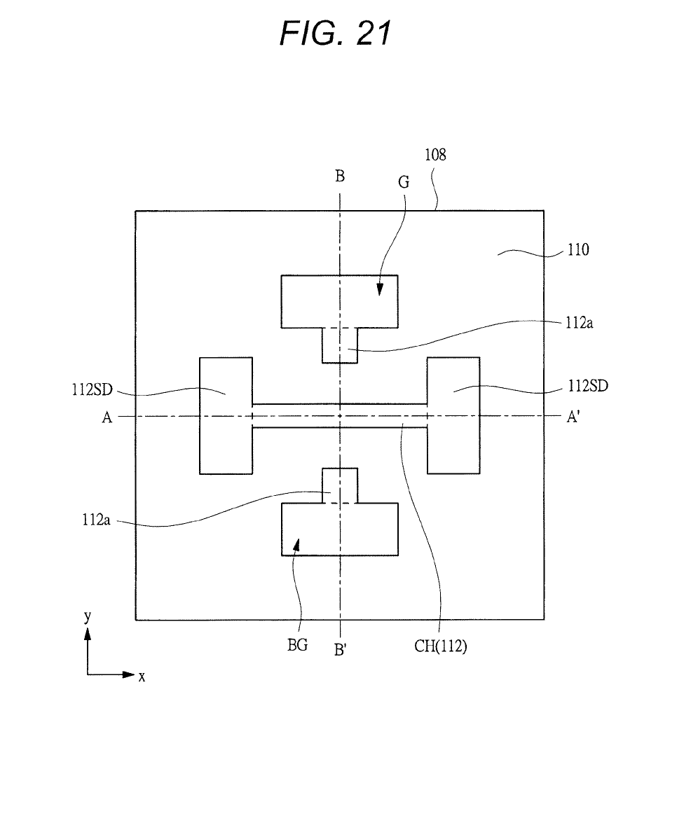

As shown in FIG. 19 through 21, patterning is then performed on the semiconductor film 112 (the polycrystalline silicon film) and the silicon oxide film 113, to form the channel region CH, the semiconductor film 112G, and the semiconductor film 112BG. This channel region CH is formed with the portion of the semiconductor film 112 (the polycrystalline silicon film) located between the source/drain regions SD. As shown in FIG. 21, the pattern (the planar shape seen from above) of the channel region CH is a rectangle having its long side extending in the x-direction. Here, the semiconductor film 112 (the polycrystalline silicon film) forming the channel region CH is patterned to have the shape of the letter H so as to cover the source/drain regions SD. The portions of the semiconductor film (the polycrystalline silicon film) located on the source/drain regions SD are denoted by 112SD. The silicon oxide film 113 is placed on this semiconductor film 112 (the polycrystalline silicon film) (see FIGS. 19 and 20).

By this patterning, the gate electrode G formed with the film stack of the semiconductor film 111G and the semiconductor film 112G located thereon is also formed. As shown in FIG. 21, the semiconductor film 112G is patterned to have a shape with a protruding portion 112a that protrudes in the direction from an upper portion of the semiconductor film 111G toward the channel region CH. That is, the semiconductor film 112G is designed to cover the upper portion of the semiconductor film 111G, the side surface of the semiconductor film 111G facing the channel region CH, and the upper surface of the silicon oxide film 110, and the portion located on the silicon oxide film 110 is the protruding portion 112a. The silicon oxide film 113 is also placed on this semiconductor film 112G (see FIG. 20).

As the gate electrode G is formed as a stack structure as described above, the side (the protruding portion 112a) facing the side surface xz1 of the channel region CH can be made thinner and sharper. The influence of a gate potential on the channel region CH, particularly on the side surface xz1, can be made greater. Accordingly, a test object can be examined with high sensitivity.

By this patterning, the back gate electrode BG formed with the film stack of the semiconductor film 111BG and the semiconductor film 112BG located thereon is also formed. As shown in FIG. 21, the semiconductor film 112BG is patterned to a shape with a protruding portion 112a that protrudes in the direction from an upper portion of the semiconductor film 111BG toward the channel region CH. That is, the semiconductor film 112BG is designed to cover the upper portion of the semiconductor film 111BG, the side surface of the semiconductor film 111BG facing the channel region CH, and the upper surface of the silicon oxide film 110, and the portion located on the silicon oxide film 110 is the protruding portion 112a. The silicon oxide film 113 is also placed on this semiconductor film 112BG (see FIG. 20).

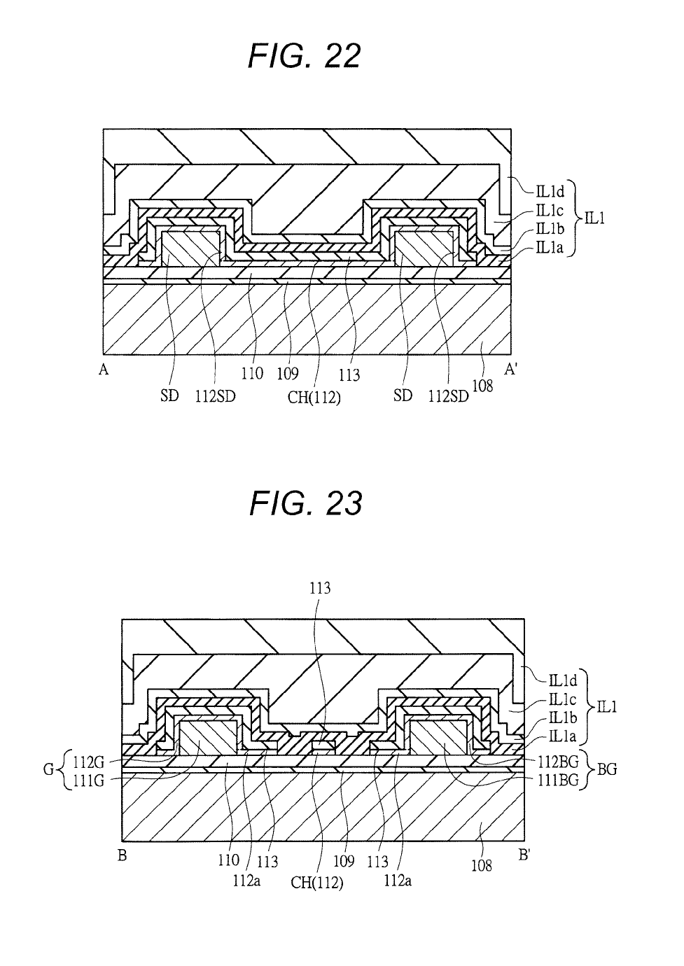

As shown in FIGS. 22 through 24, an interlayer insulating film IL1 is formed on the underlayer insulating film (the silicon oxide film 110) as well as on the silicon oxide film 113. The interlayer insulating film IL1 is a film stack formed by stacking a silicon oxide film IL1a, a silicon nitride film IL1b, a silicon oxide film IL1c, and a silicon nitride film IL1d in this order from the bottom, for example. Specifically, the silicon oxide film IL1a of approximately 20 nm in thickness is deposited on the silicon oxide film 113 by CVD or the like, and the silicon nitride film IL1b of approximately 10 nm in thickness is then deposited thereon by CVD or the like. Further, the silicon oxide film IL1c of approximately 150 nm in thickness is deposited by CVD or the like, and the silicon nitride film IL1d of approximately 200 nm in thickness is then deposited thereon by CVD or the like. For example, the silicon oxide film IL1a exists between the protruding portion 112a of the gate electrode G and the channel region CH, and serves as a gate insulating film. The silicon oxide film IL1a also exists between the protruding portion 112a of the back gate electrode BG and the channel region CH.

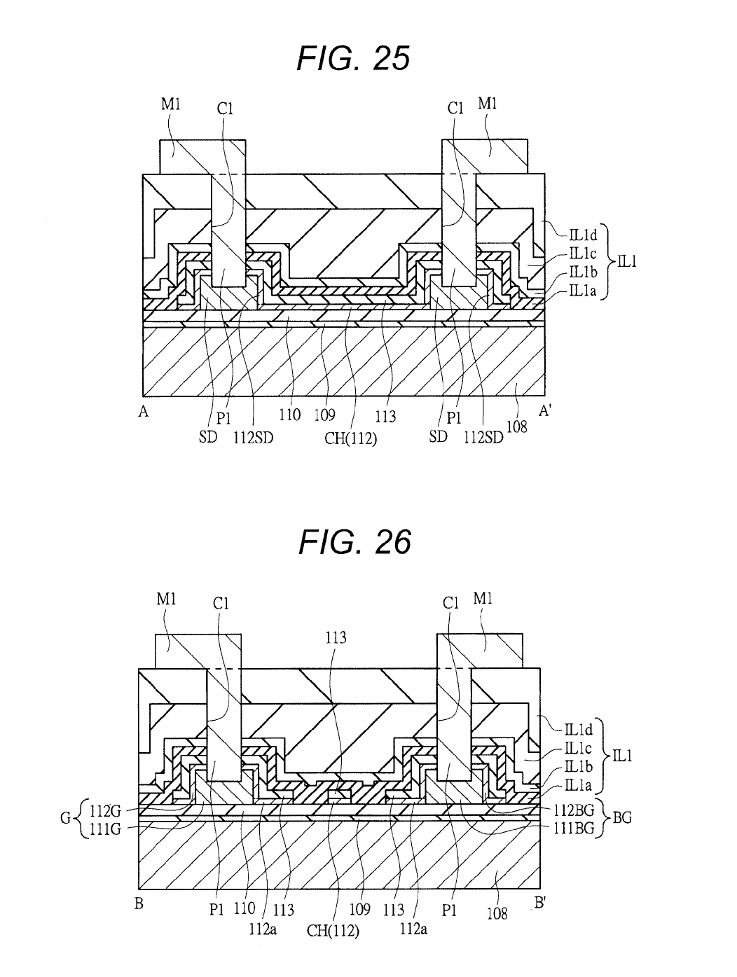

As shown in FIGS. 25 through 27, patterning is performed on the interlayer insulating film IL1 and the semiconductor films (112SD, 112G, 112BG, and the polycrystalline silicon film), to form contact holes C1 that reach the source/drain regions SD, the gate electrode G, and the back gate electrodes BG. A conductive film is then formed on the interlayer insulating film IL1 as well as in the contact holes C1. The conductive film may be a metal film such as an aluminum (Al) film. For example, the aluminum film is deposited on the interlayer insulating film IL1 as well as in the contact holes C1 by a sputtering technique or the like, and patterning is then performed on the aluminum film, to form first plugs (connecting portions) P1 and first interconnect layers M1.

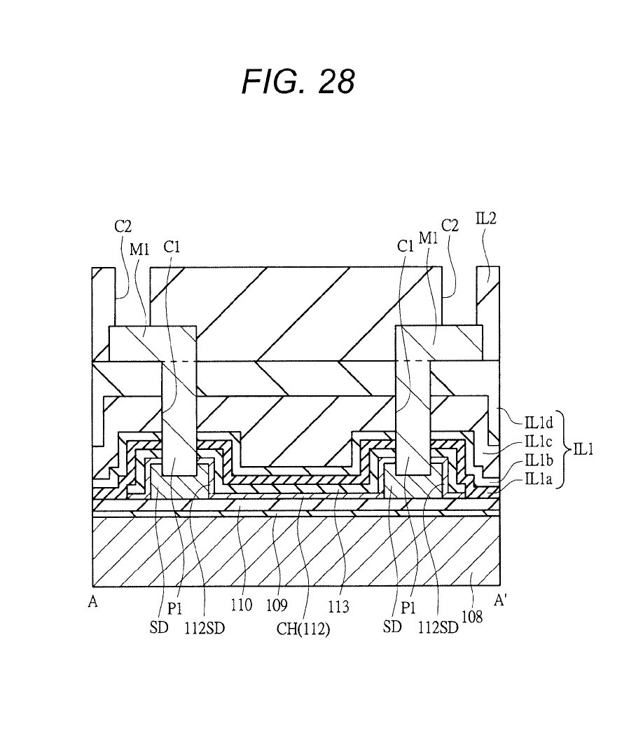

As shown in FIGS. 28 through 30, a silicon nitride film, for example, is formed as an interlayer insulating film IL2 on the interlayer insulating film IL1 as well as on the first interconnect layers M1. The silicon nitride film of approximately 200 nm is deposited by CVD, for example. Patterning is then performed on the interlayer insulating film IL2, to form contact holes C2 that reach the first interconnect layers M1. The contact holes C2 are to be electrical connecting portions for the source/drain regions SD, the gate electrode G, and the back gate electrode BG. The contact holes C2 may be filled with conductive films, to form terminals. Alternatively, external connection terminals may be inserted.

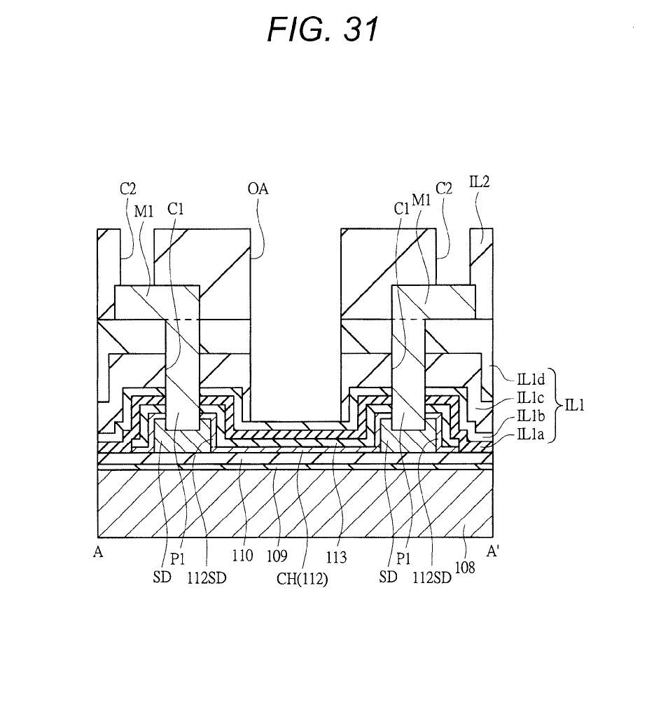

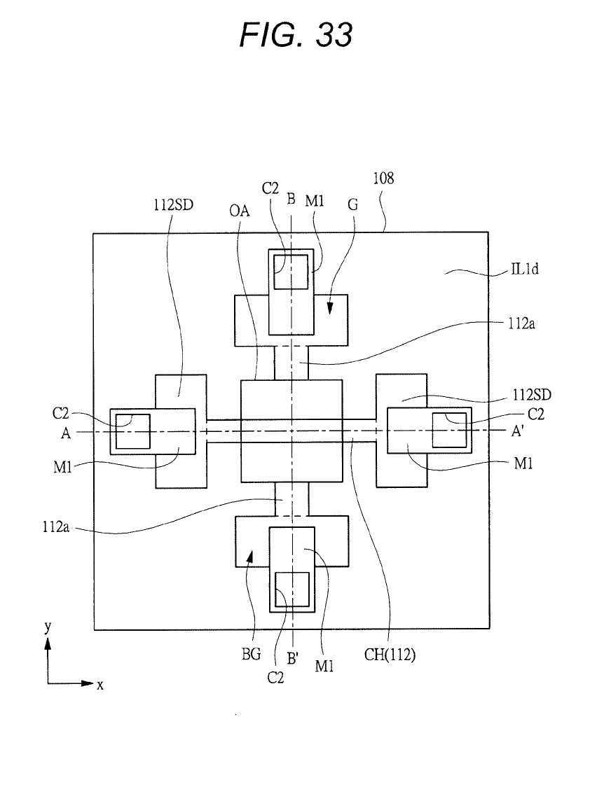

As shown in FIGS. 31 through 33, patterning is then performed on portions of the interlayer insulating film IL2 and the interlayer insulating film IL1 located above the channel region CH, to form an opening OA. For example, etching is performed on the interlayer insulating film IL2, and the silicon nitride film IL1d and the silicon oxide film IL1c, which are the two upper layers in the interlayer insulating film IL1. As the etching is performed on the interlayer insulating films (IL2 and IL1) above the channel region CH, the films stacked above the channel region CH are made thinner. After this procedure, the silicon nitride film IL1b, the silicon oxide film IL1a, and the silicon oxide film 113, which are relatively thin, remain on the channel region CH. The pattern (the planar shape seen from above) of the opening OA is a rectangle that includes the center portion and its surrounding area of the channel region CH (FIG. 33). The opening OA is designed to include the region in which the later described pore P is to be formed.

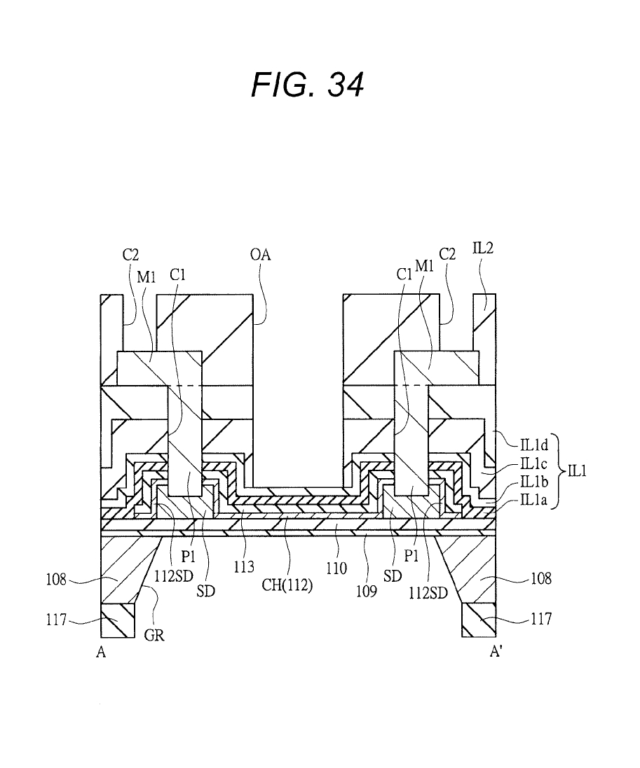

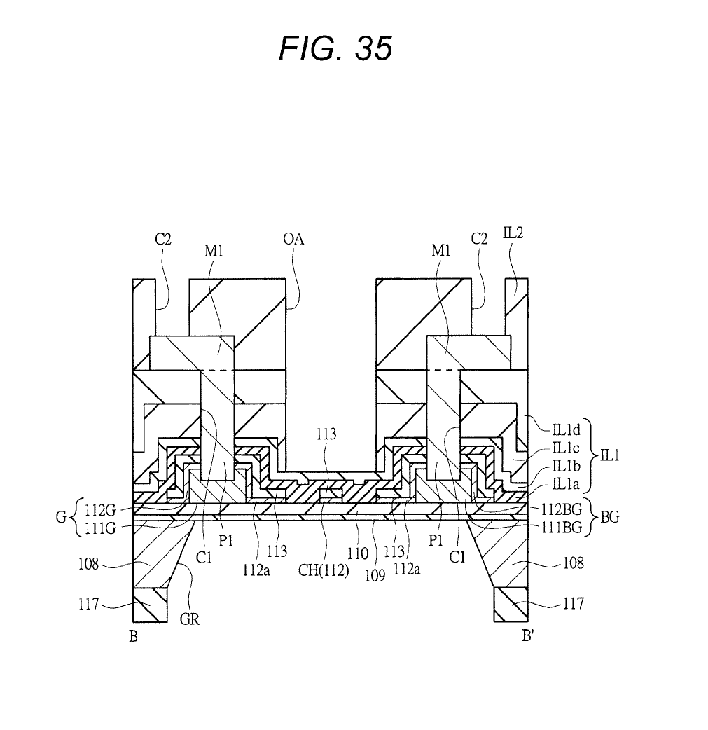

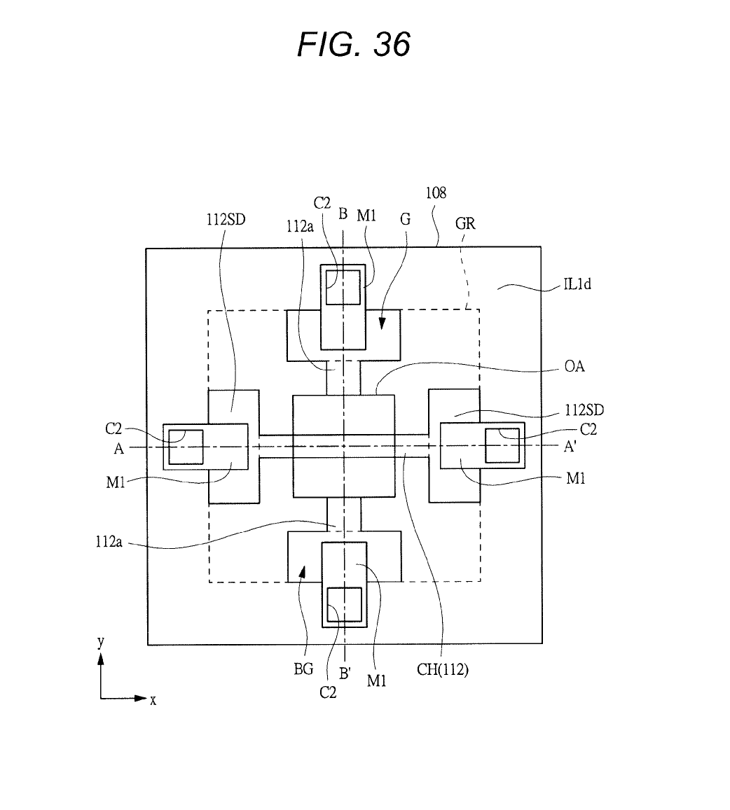

As shown in FIGS. 34 through 36, a silicon nitride film, for example, is formed as a hard mask 117 on the bottom surface of the supporting substrate 108. This silicon nitride film is deposited by CVD, for example. Patterning is then performed on the hard mask 117, to form a hard mask 117 having an opening below the channel region CH. With the hard mask 117 being a mask, etching is performed on the supporting substrate (silicon substrate) 108, to form a groove GR. For example, wet etching using a KOH (potassium hydroxide) solution or a TMAH solution is performed on the bottom surface of the supporting substrate 108. In this manner, the portion of the supporting substrate 108 located below the channel region CH is made thinner. Here, the groove GR is formed by performing etching on the supporting substrate 108 until the silicon nitride film 109 is exposed. After this procedure, the silicon oxide film 110 and the silicon nitride film 109, which are relatively thin, remain under the channel region CH. For example, the pattern (the planar shape seen from above) of the groove GR is a rectangle that includes the center portion and its surrounding area of the channel region CH, as shown in FIG. 36. The groove GR is designed to include the region in which the later described pore P is to be formed. Here, the pattern of the groove GR corresponds to a relatively wide region that includes not only the channel region CH but also the formation regions of the source/drain regions SD, the gate electrode G, and the back gate electrode BG.

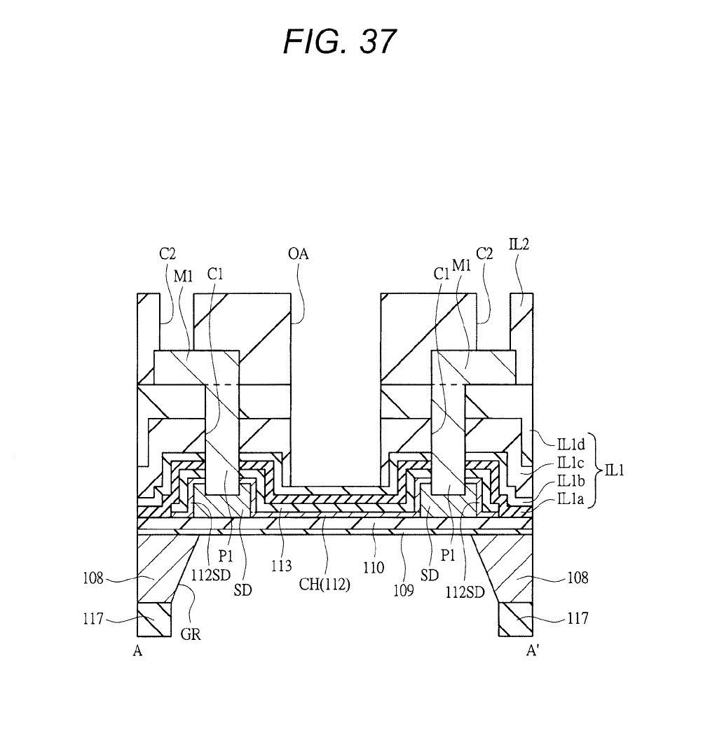

As shown in FIGS. 37 through 39, the pore (the hole, the through hole, or the opening) P is then formed. In the opening OA, a region between the channel region CH and the upper semiconductor film 112G on the gate electrode G is irradiated with an energy beam such as a TEM beam, to form the pore P penetrating through the silicon nitride film IL1b, the silicon oxide film IL1a, the silicon oxide film 110, and the silicon nitride film 109. The diameter of the pore P is preferably 5 nm or smaller. With an energy beam, the pore P having a very small diameter can be readily formed. Since the films and substrate located above and below the channel region CH and its surrounding region have already been made thinner as described above, the minute pore P can be formed with high controllability.

The pore P may be formed not with the above described energy beam but by etching. For example, etching may be performed on the silicon nitride film IL1b, the silicon oxide film IL1a, the silicon oxide film 110, and the silicon nitride film 109, to form the through hole to be the pore P. If the diameter of the through hole is large, a silicon oxide film may be formed by CVD or the like on the supporting substrate 108 as well as in the through hole, to reduce the diameter of the through hole. In that case, the silicon oxide film is also formed on the sidewall of the through hole, and accordingly, the diameter of the pore P can be made smaller. In a case where the through hole is formed with a TEM beam or the like, the hole diameter may of course be adjusted by forming a silicon oxide film on the sidewall of the through hole as described above.

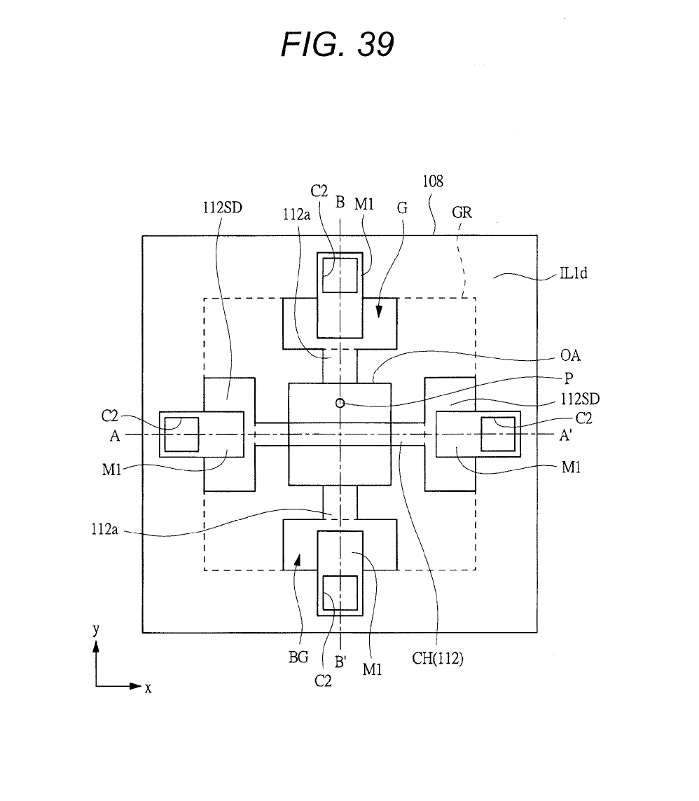

As shown in FIG. 39, the pore P is formed in the opening OA and in a region between the channel region CH and the gate electrode G.

As the pore P is located in such a position, influence of the nucleotides constituting the DNA passing through the pore P on the source-drain current can be made larger. Specifically, influence of each nucleotide on the quasi-one-dimensional thin current path (the inversion layer 10) formed in the side surface xz1 of the channel region CH can be made larger (see FIGS. 2(A) and 2(B)). Accordingly, the source-drain current can be made to greatly vary depending on the types of nucleotides passing through the pore P. Thus, the respective nucleotides constituting DNA can be detected with high sensitivity, and a genomic sequence can be efficiently analyzed.

### Second Embodiment

Modifications of the location position of the pore P of the first embodiment are now described in this embodiment.

As described in the first embodiment, the inversion layer (the channel) 10 is formed with electrons (carriers) induced by an electric field from the gate electrode G (see FIGS. 2(A) and 2(B)). The position of the pore P is preferably between the inversion layer 10 and the gate electrode G. As the pore P is located between them, changes in potential due to a test object introduced into the pore P can be effectively reflected by the source-drain current (the channel current). For example, if the pore P is located between the inversion layer 10 and the back gate electrode BG, changes in the source-drain current (the channel current) become much smaller.

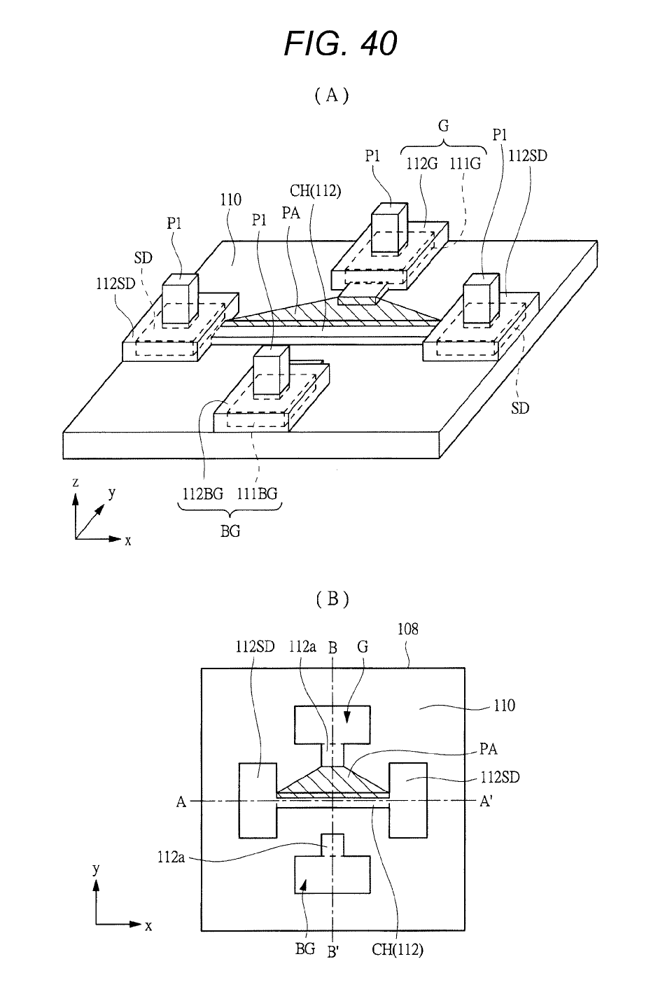

FIGS. 40(A) and 40(B) are a perspective view and a plan view showing a preferred region in which the pore P is to be located. FIG. 40(A) is the perspective view, and FIG. 40(B) is the plan view. The preferred region PA in which the pore P is to be located is indicated by a shaded region. As shown in FIGS. 40(A) and 40(B), it is preferable to form the pore P in the shaded region PA located between the side surface (xz1) of the channel region CH and the side surface of the gate electrode G facing the side surface xz1. As the distance between the inversion layer and the pore P in the region PA becomes shorter, detection sensitivity becomes higher. The inversion layer (the channel) is formed inside the side surface xz1 of the channel region CH (see FIGS. 2(A) and 2(B)). Therefore, the pore P is preferably formed in a position as close to the side surface xz1 of the channel region CH as possible. For example, the pore P may be formed in the insulating film (the silicon oxide film IL1a) located between the gate electrode G and the channel region CH as described in the first embodiment (see FIG. 1 and others), in a boundary portion between the channel region CH and the insulating film (the silicon oxide film IL1a), or inside the side surface xz1 of the channel region CH. Therefore, the preferred region PA in which pore P is to be located includes the region that extends parallel to the side surface xz1 of the channel region CH and has a predetermined width (approximately 15 nm, for example).

(Modification 1)

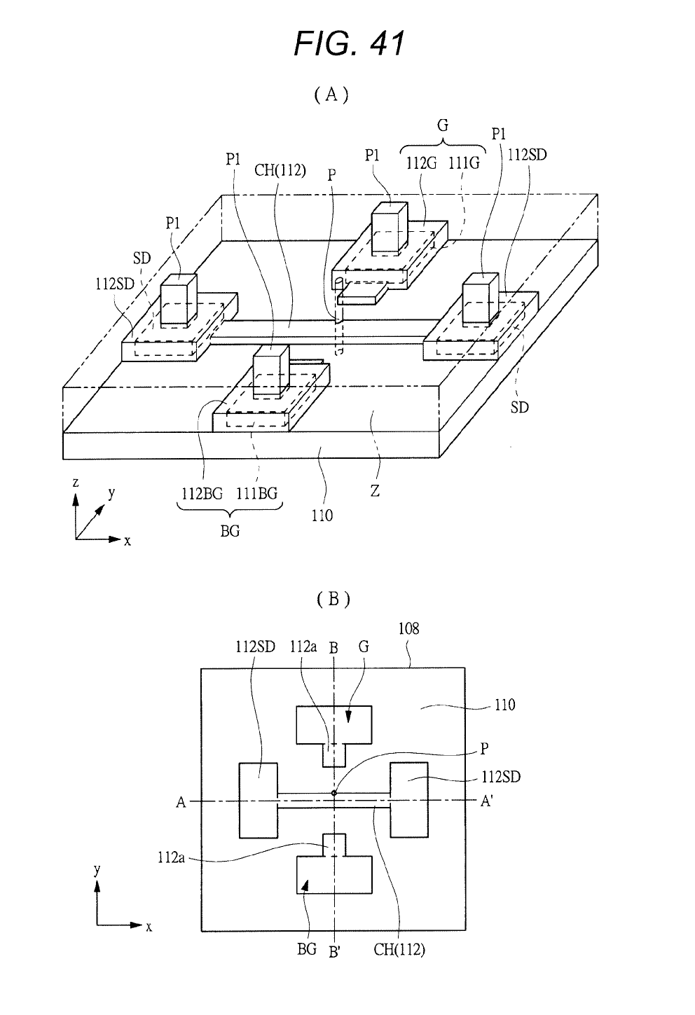

FIGS. 41(A) and 41(B) are a perspective view and a plan view of a structure according to Modification 1 of the semiconductor device of this embodiment. FIG. 41(A) is the perspective view, and FIG. 41(B) is the plan view. As shown in FIGS. 41(A) and 41(B), in Modification 1, the pore P is located in a boundary portion between the side surface xz1 of the channel region CH and the insulating film (the silicon oxide film IL1a). For example, the pore P is located so that a half of the pore P is located in the side surface xz1 of the channel region CH and the other half is located in the insulating film (the silicon oxide film IL1a). The other aspects of the structure are the same as those of the first embodiment, and therefore, detailed explanation of them is not repeated herein.

As the pore P is located as described above, the inversion layer (the channel) can be formed in contact with the pore P, and detection sensitivity can be increased.

(Modification 2)

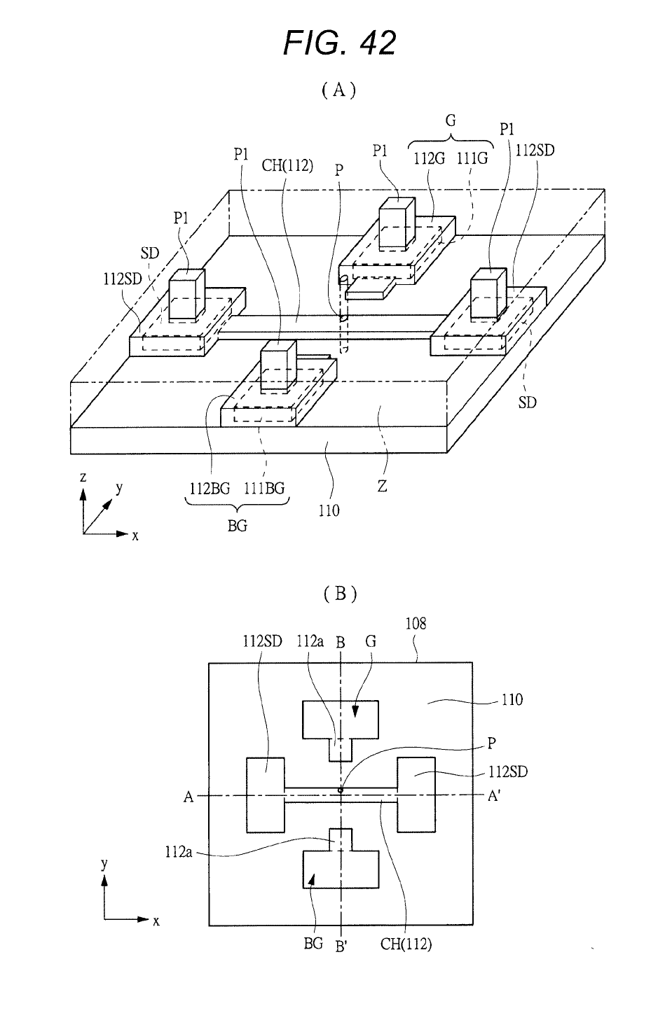

FIGS. 42(A) and 42(B) are a perspective view and a plan view of a structure according to Modification 2 of the semiconductor device of this embodiment. FIG. 42(A) is the perspective view, and FIG. 42(B) is the plan view. As shown in FIGS. 42(A) and 42(B), in Modification 2, the pore P is located inside the side surface xz1 of the channel region CH. For example, the pore P is located in the channel region CH, and a side portion of the pore P is in contact with the side surface xz1 of the channel region CH. The other aspects of the structure are the same as those of the first embodiment, and therefore, detailed explanation of them is not repeated herein.

As the pore P is located as described above, the inversion layer (the channel) can be formed in contact with the pore P, and detection sensitivity can be increased.

A method of operating the semiconductor device of this embodiment (Modification 1 or 2) is the same as that of the first embodiment, and therefore, detailed explanation of it is not repeated herein. That is, as in the first embodiment, a test object such as DNA is introduced into the pore P, and the sequence of nucleotides constituting the DNA is analyzed by detecting changes in current between the source/drain regions SD.

As described above, according to this embodiment, detection sensitivity can be made higher than that of the first embodiment. However, in the structure of the first embodiment (see FIG. 1 and others), any test object is not in contact with the channel region CH. In other words, a test object (a solution, for example) passes through an insulating film (the silicon oxide film IL1a). Accordingly, characteristics degradation of the channel region CH, such as oxidation and corrosion of the channel region CH due to a test object (a solution, for example), can be reduced. Also, there is no need to take into consideration oxidation resistance and corrosion resistance of the channel region CH, and accordingly, a material can be selected from a wider variety of options. Furthermore, there is no need to perform an antioxidation treatment or an anticorrosion treatment on the channel region CH, and accordingly, the manufacturing procedures are simplified.

A method of manufacturing the semiconductor device of this embodiment (Modification 1 or 2) is the same as that of the first embodiment, except for the procedures for forming the pore P. Therefore, detailed explanation of the method is not repeated herein. For example, the position in which the pore P is to be formed should be changed in the pore forming procedures described above with reference to FIGS. 37 through 39 in the first embodiment. Specifically, the pore P is located in a boundary portion between the channel region CH and an insulating film (the silicon oxide film IL1a), or inside the side surface xz1 of the channel region CH. For example, the portion in such a position is irradiated with an energy beam such as a TEM beam, to form the pore P penetrating through the silicon nitride film IL1b, the silicon oxide film IL1a, the silicon oxide film 113, the channel region CH (112), the silicon oxide film 110, and the silicon nitride film 109.

### Third Embodiment

Modifications of the shapes of the channel region CH, the gate electrode G, the back gate electrode BG, and the semiconductor films 112SD of the first embodiment are now described in this embodiment.

(Modification A)

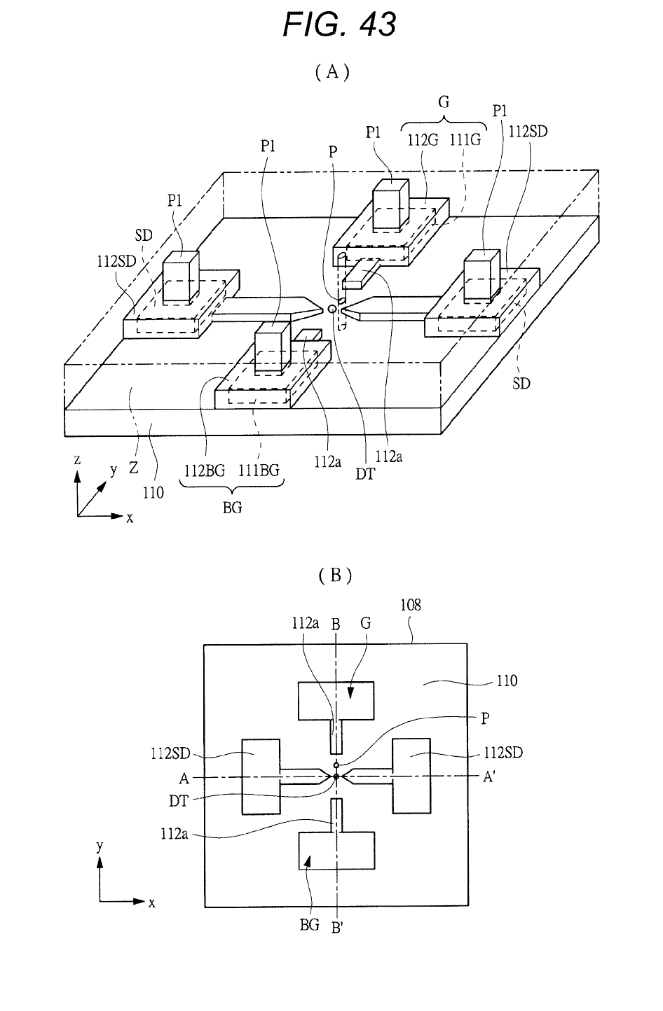

FIGS. 43(A) and 43(B) are a perspective view and a plan view of a structure according to Modification A of the semiconductor device of this embodiment. FIG. 43(A) is the perspective view, and FIG. 43(B) is the plan view.

As shown in FIGS. 43(A) and 43(B), in Modification A, the channel region CH is formed with a Si (silicon) dot DT. A so-called “single-electron transistor” is used. As single electrons pass through this Si dot (a Coulomb island or a quantum dot) DT, current flows between the source/drain regions SD.

Whether or not current flows between the source/drain regions SD via the Si dot DT is controlled by an electric field generated from the gate electrode G toward the Si dot DT. As a result, potential changes occur due to a test object in the pore P, and accordingly, the permission/prohibition or probability of traveling of single electrons to the Si dot DT changes.

As the Si dot DT is used as the channel region as described above, the existence/nonexistence or changes of a test object in the pore P can be dramatically well reflected by the rate of change in detection signals. Accordingly, high-sensitivity detection can be performed.

As shown in FIGS. 43(A) and 43(B), the semiconductor device of this embodiment includes the source/drain regions SD and the gate electrode (a control gate electrode) G, which are placed on an insulating film (an insulating layer) such as the silicon oxide film 110. The Si dot DT is placed between the source/drain regions SD. The semiconductor films (polycrystalline silicon films) 112SD on the source/drain regions SD extend from an upper portion of the source/drain regions SD to the vicinity of the Si dot DT. The ends thereof are triangular in shape in the plan view seen from above.

The distance between each semiconductor film 112SD and the Si dot DT is approximately 1 to 10 nm, for example, and the distance between the Si dot DT and the pore P is approximately 1 to 5 nm, for example. Operations of the semiconductor device of this embodiment are the same as those of the first embodiment.

(Modification B)

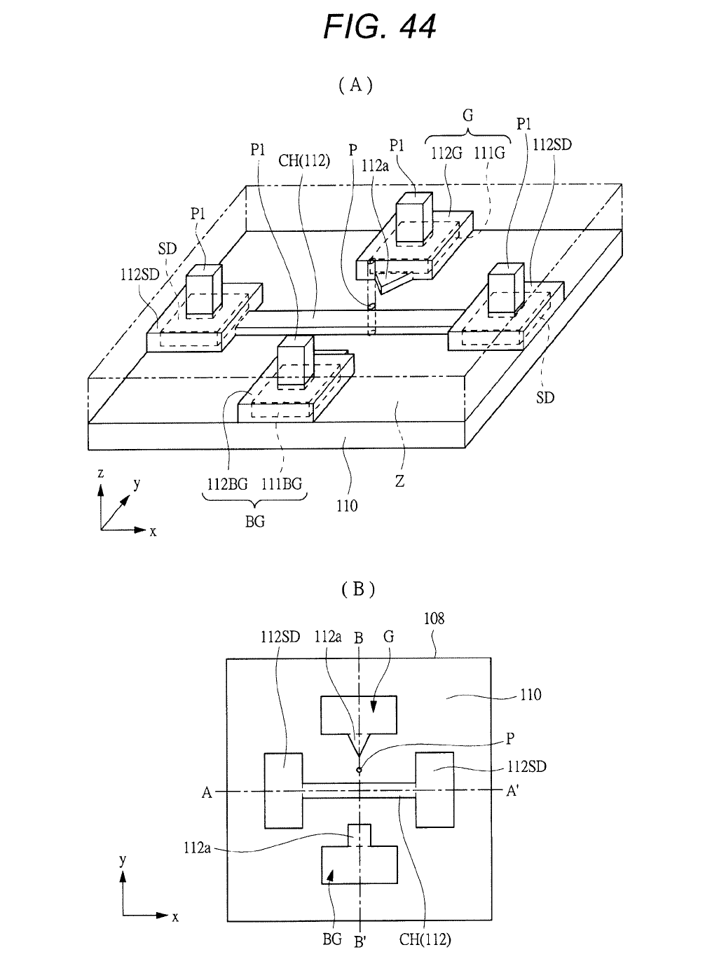

FIGS. 44(A) and 44(B) are a perspective view and a plan view of a structure according to Modification B of the semiconductor device of this embodiment. FIG. 44(A) is the perspective view, and FIG. 44(B) is the plan view.

As shown in FIGS. 44(A) and 44(B), in Modification B, the shape of the protruding portion 112a of the gate electrode G (in a plan view seen from above) is triangular.

Specifically, the semiconductor film 112G of the gate electrode G, which is formed with a film stack of the semiconductor film 111G and the upper semiconductor film 112G, extends in the direction from an upper portion of the semiconductor film 111G toward the channel region CH, and the end portion thereof is triangular in shape. In other words, the semiconductor film 1126 has a triangular protruding portion 112a. More specifically, of the semiconductor film 112G, the portion located on the silicon oxide film 110 has a shape including the triangular protruding portion 112a.

As the protruding portion 112a has a triangular shape with its sharpened portion protruding toward the pore P as described above, the voltage applied to the gate electrode G is intensified at the end portion, and the electric field to be applied to the pore P becomes stronger. Accordingly, influence of a test object in the pore P on the electric field can be clearly detected as changes in the current between the source/drain regions SD (the channel current).

The semiconductor device of this embodiment has the same structure as the semiconductor device of the first embodiment, except for the shape of the semiconductor film 112G, and therefore, explanation of the structure is not repeated herein. Operations are also the same as those of the first embodiment. The manufacturing procedures are also the same as those of the first embodiment, except that the protruding portion 112a is designed to have a triangular shape at the time of patterning of the semiconductor film 112.

(Modification C)

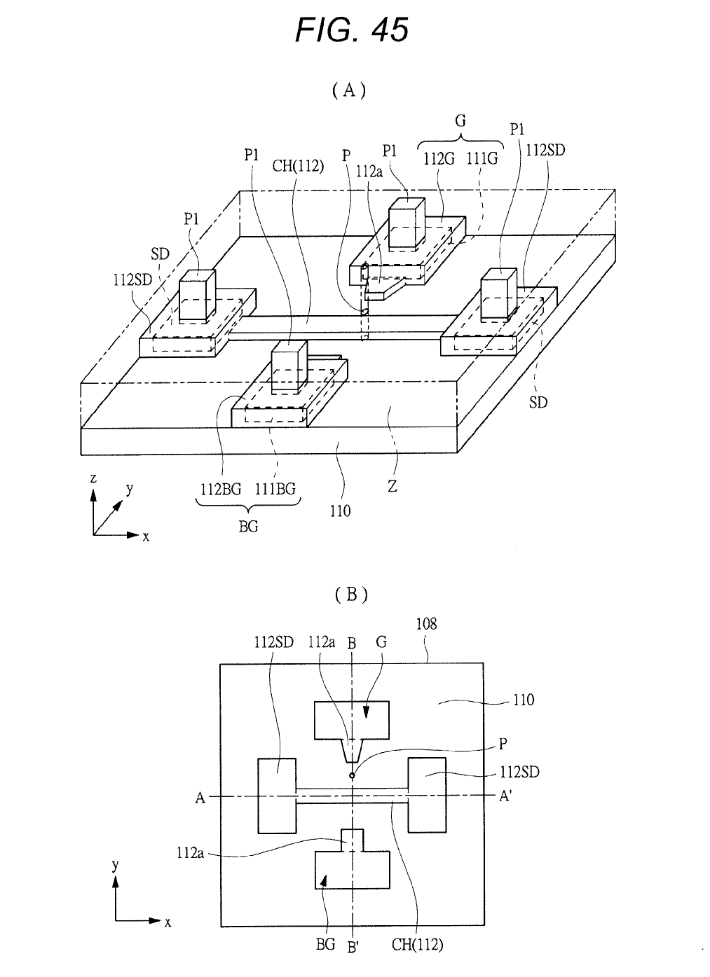

FIGS. 45(A) and 45(B) are a perspective view and a plan view of a structure according to Modification C of the semiconductor device of this embodiment. FIG. 45(A) is the perspective view, and FIG. 45(B) is the plan view.

As shown in FIGS. 45(A) and 45(B), in Modification C, the shape of the protruding portion 112a of the gate electrode G (in a plan view seen from above) is trapezoidal. Of the upper and lower bases of the trapezoidal shape, the shorter base is located on the side of the channel region CH, and the longer base is located on the side of the semiconductor film 111G.

Specifically, the semiconductor film 112G of the gate electrode G, which is formed with a film stack of the semiconductor film 111G and the upper semiconductor film 112G, extends in the direction from an upper portion of the semiconductor film 111G toward the channel region CH, and the end portion thereof has a trapezoidal shape. In other words, the semiconductor film 112G has a trapezoidal protruding portion 112a.

As the protruding portion 112a has a trapezoidal shape with its sharpened portion protruding toward the pore P as described above, the voltage applied to the gate electrode G is intensified at the end portion, and the electric field to be applied to the pore P becomes stronger. Accordingly, influence of a test object in the pore P on the electric field can be clearly detected as changes in the current between the source/drain regions SD (the channel current).

The semiconductor device of this embodiment has the same structure as the semiconductor device of the first embodiment, except for the shape of the semiconductor film 112G, and therefore, explanation of the structure is not repeated herein. Operations are also the same as those of the first embodiment. The manufacturing procedures are also the same as those of the first embodiment, except that the protruding portion 112a is designed to have a trapezoidal shape at the time of patterning of the semiconductor film 112.

(Modification D)

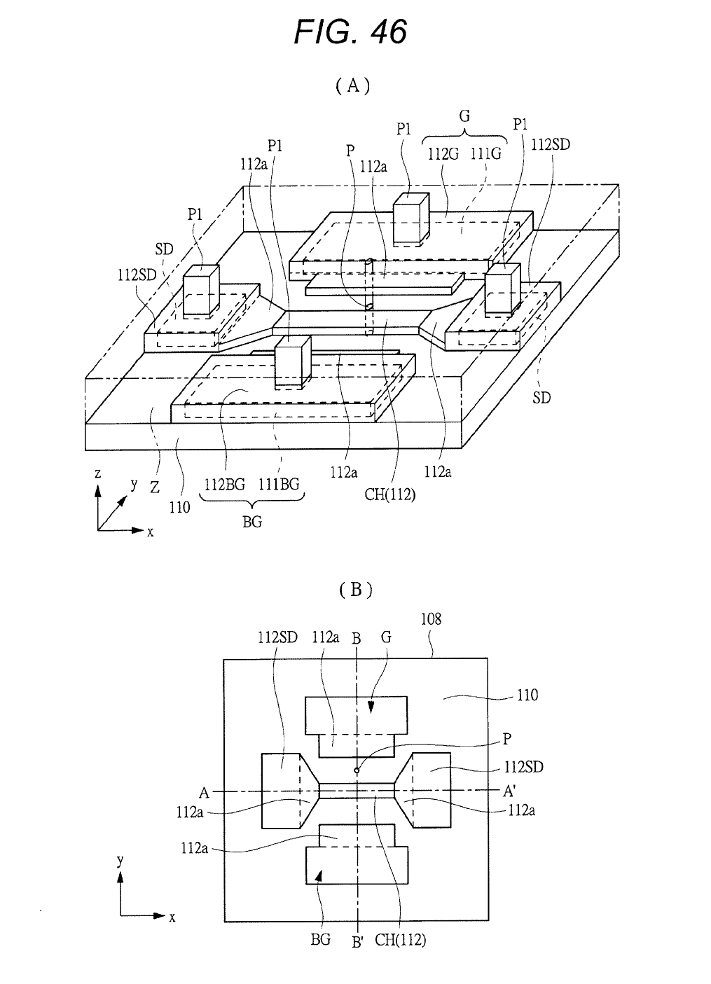

FIGS. 46(A) and 46(B) are a perspective view and a plan view of a structure according to Modification D of the semiconductor device of this embodiment. FIG. 46(A) is the perspective view, and FIG. 46(B) is the plan view.

As shown in FIGS. 46(A) and 46(B), in Modification D, each semiconductor film 112SD has a protruding portion 112a, and the shape of this protruding portion 112a (in a plan view seen from above) is trapezoidal. Of the upper and lower bases of the trapezoidal shape, the shorter base is located on the side of the channel region CH, and the longer base is located on the side of the source/drain region SD. In this case, the semiconductor films 112SD are regarded as included in the source/drain regions SD. In Modification D, the gate length of the gate electrode G (the length of the gate electrode G in the x-direction) is greater than that of the first embodiment. The gate length of the back gate electrode BG (the length of the back gate electrode BG in the x-direction) is also greater than that of the first embodiment.

With this arrangement, the facing areas of the gate electrode G and the channel region CH can be made larger. Accordingly, the electric field from the gate electrode G can be efficiently applied to the entire surface of the channel region CH, and transistor characteristics can be improved. Specifically, the channel current value, the S value, and the like can be improved.

Thus, the detection current value is increased, and detection signal processing becomes easier. Also, the signal processing speed can be increased. In a case where a threshold voltage shift occurs due to influence of a test object in the pore P, a detection current shift amount at a certain gate voltage can be made larger by virtue of the improved S value, and discrimination ability (detection sensitivity) can be increased.

The semiconductor device of this embodiment has the same structure as the semiconductor device of the first embodiment, except for the pattern shapes of the semiconductor films 112SD, the gate electrode G, and the back gate electrode BG, and therefore, explanation of the structure is not repeated herein. Operations are also the same as those of the first embodiment. The manufacturing procedures are also the same as those of the first embodiment, except that the semiconductor films 112G, 112BG, and 112SD are patterned to have the above described shapes.

(Modification E)

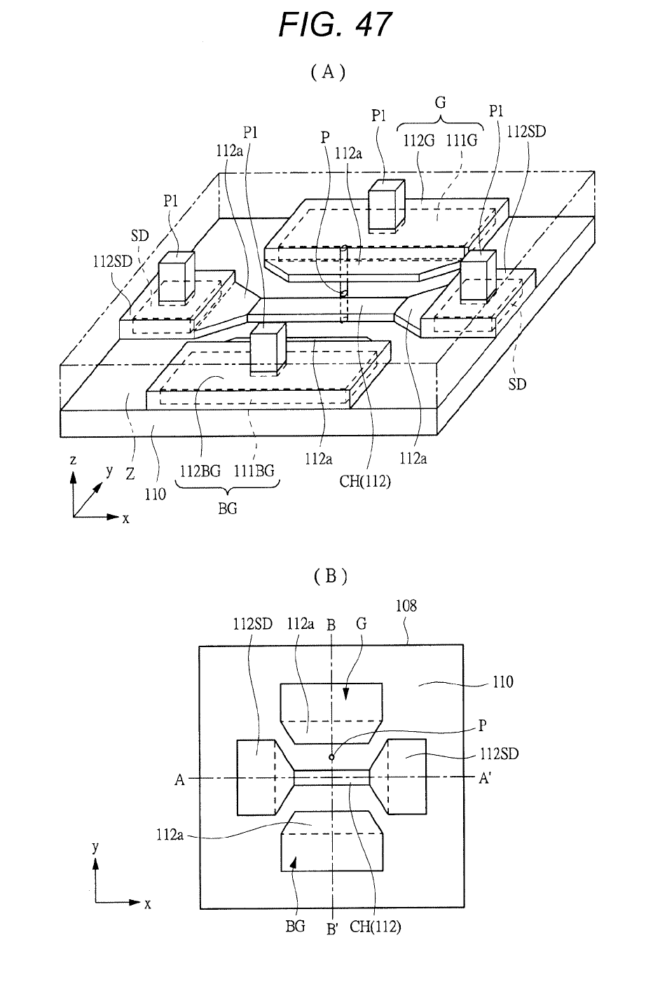

FIGS. 47(A) and 47(B) are a perspective view and a plan view of a structure according to Modification E of the semiconductor device of this embodiment. FIG. 47(A) is the perspective view, and FIG. 47(B) is the plan view.

As shown in FIGS. 47(A) and 47(B), in Modification E, each semiconductor film 112SD has a protruding portion 112a, and the shape of this protruding portion 112a (in a plan view seen from above) is trapezoidal, as in Modification D described above. Of the upper and lower bases of the trapezoidal shape, the shorter base is located on the side of the channel region CH, and the longer base is located on the side of the source/drain region SD, as in the above modification.

In Modification E, the gate lengths of the gate electrode G and the back gate electrode BG (the lengths of the respective electrodes in the x-direction) are longer than those of the first embodiment, as in Modification D described above.

Modification E differs from Modification D in that the semiconductor film 112G of the gate electrode G has a shape including a trapezoidal protruding portion 112a. The semiconductor film 112BG of the back gate electrode BG also has a shape including a trapezoidal protruding portion 112a.

As the end portion of the gate electrode G has a trapezoidal shape as described above, electric field concentration at the edge portions (corner portions) of the gate electrode G can be relaxed. Likewise, electric field concentration at the edge portions of the back gate electrode BG can be relaxed. Accordingly, the withstand voltage of the semiconductor device can be increased.

In this manner, not only the effects of Modification D described above but also an increase in withstand voltage can be achieved.

The semiconductor device of this embodiment has the same structure as the semiconductor device of the first embodiment, except for the shapes of the semiconductor films 112G, 112BG, and 112SD, and therefore, explanation of the structure is not repeated herein. Operations are also the same as those of the first embodiment. The manufacturing procedures are also the same as those of the first embodiment, except that the semiconductor films 112G, 112BG, and 112SD are patterned to have the above described shapes at the time of patterning of the semiconductor film 112.

### Fourth Embodiment

Referring to drawings, the structure of a semiconductor device of this embodiment and a method of manufacturing the semiconductor device will be described below in detail.

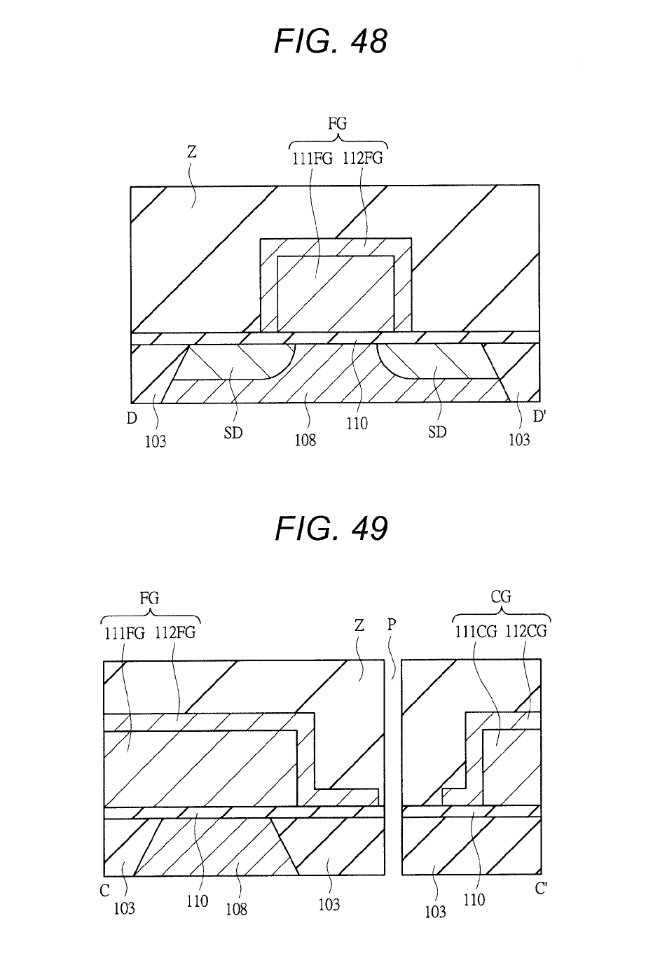

FIGS. 48 through 50 are cross-sectional views and a plan view of relevant components of the semiconductor device of this embodiment. Each cross-sectional view is equivalent to the C-C′ cross-section or the D-D′ cross-section of the plan view.

As shown in FIGS. 48 through 50, the semiconductor device of this embodiment includes a floating gate electrode FG and a control gate electrode CG, which are placed on a first surface (the outer surface or the upper surface) of an insulating film (an insulating layer) such as a silicon oxide film 110.

A side surface of the floating gate electrode FG and a side surface of the control gate electrode CG are arranged to face each other, and a pore (a hole, a through hole, or an opening) P is formed in a region between (the side surfaces of) the floating gate electrode FG and the control gate electrode CG. This pore P is designed to penetrate through an insulating film Z, the silicon oxide film 110, and the like. The pore P is also designed to extend parallel to the side surface of the floating gate electrode FG and is perpendicular to the first surface of the silicon oxide film 110. The pore P is a hole (an opening) through which a test object such as a bio-related substance like DNA passes. The diameter of the pore P may be adjusted in accordance with the size of the test object, and is preferably not smaller than 1 nm and not greater than 5 nm when a bio-related substance such as DNA passes therethrough. Since the thickness of DNA is approximately 1 nm, the diameter of the pore P is preferably not smaller than 1 nm, and is preferably not greater than 5 nm so as to examine a test object with high sensitivity.

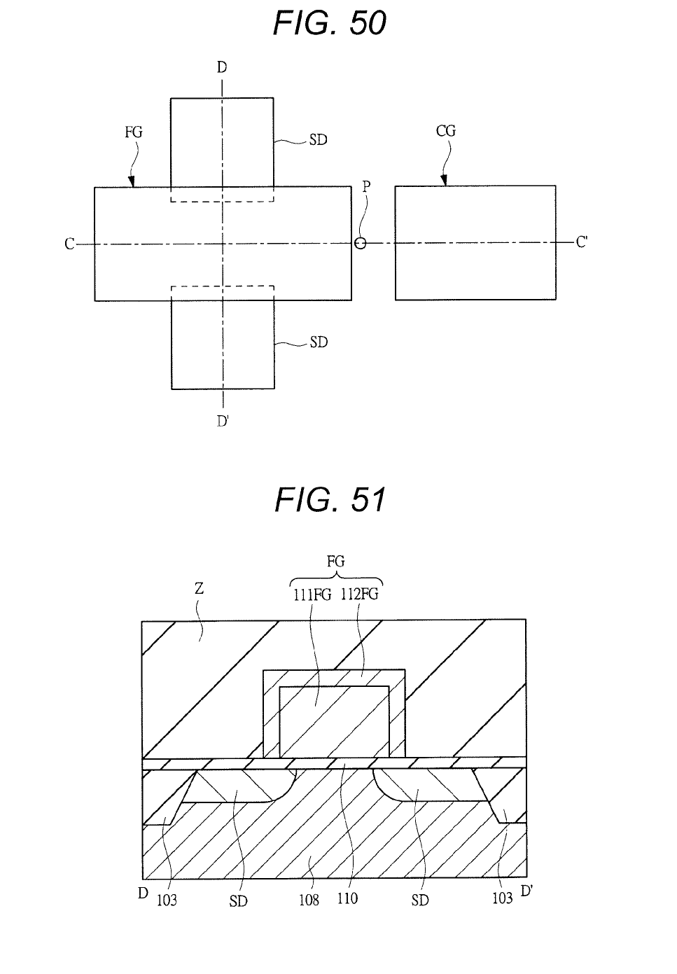

Source/drain regions SD are formed on a second surface (the bottom surface or the lower surface) of the silicon oxide film 110 (FIG. 48). As shown in FIG. 50, the source/drain regions SD are placed on both sides of the floating gate electrode FG in the direction perpendicular to the extending direction (the vertical direction in the drawing) of the floating gate electrode FG. With this arrangement, the semiconductor device of this embodiment has a FET structure including the floating gate electrode FG and the source/drain regions SD. Reference numeral 103 indicates a device isolation insulating film.

The floating gate electrode FG is formed with a film stack of an n-type semiconductor film 111FG and a semiconductor film 112FG (FIG. 49). The semiconductor film 112FG is thinner than the semiconductor film 111FG. The semiconductor film 112FG covers the upper portion of the semiconductor film 111FG and the side surface of the semiconductor film 111FG facing the control gate electrode CG, and extends onto the upper surface of the silicon oxide film 110. As the floating gate electrode FG is formed as a stack structure as described above, the thickness of the side surface facing the pore P can be made smaller, and influence of a gate potential on the pore P can be made greater. Accordingly, a test object can be examined with high sensitivity.

The gate electrode CG is formed with a film stack of an n-type semiconductor film 111CG and a semiconductor film 112CG. The semiconductor film 112CG is thinner than the semiconductor film 111CG. The semiconductor film 112CG covers the upper portion of the semiconductor film 111CG and the side surface facing the floating gate electrode FG, and extends onto the upper surface of the silicon oxide film 110. As the control gate electrode CG is formed as a stack structure as described above, the thickness of the side surface facing the pore P can be made smaller, and influence of a gate potential on the pore P can be made greater. Accordingly, a test object can be examined with high sensitivity.

A potential difference is generated between the source/drain regions SD, so that current can flow between the source/drain regions SD by virtue of voltage application to the control gate electrode CG. At this point, the current between the source/drain regions SD is changed by the applied voltage.

Accordingly, when the electric field between the control gate electrode CG and the floating gate electrode FG is modulated by the field change caused by a test object in the pore P, the potential of the control gate electrode CG changes with the field change. The current between the source/drain regions SD also changes accordingly. Thus, the test object can be analyzed.

In the above described structure, the area of the FET formed with the source/drain regions SD and the control gate electrode CG can be easily made larger. Accordingly, the value of the current between the source/drain regions SD of the FET, or the value of the detection current, can be made greater. As the detection current becomes higher, detection signal processing becomes easier, and the speed of detection signal processing can be increased.

Referring now to FIGS. 51 through 54, a method of manufacturing the semiconductor device of this embodiment is described, and the structure of this semiconductor device is described in greater detail. FIGS. 51 through 54 are cross-sectional views of relevant components, illustrating the procedures for manufacturing the semiconductor device of this embodiment. Each cross-sectional view is equivalent to the C-C′ cross-section or the D-D′ cross-section of the plan view shown in FIG. 50.

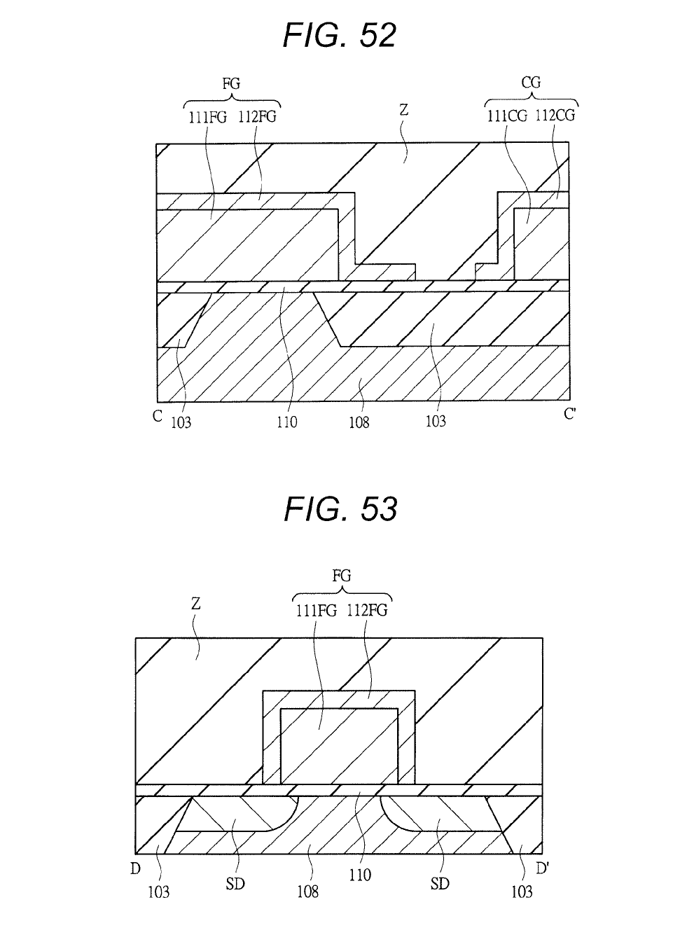

First, as shown in FIGS. 51 and 52, a silicon substrate, for example, is prepared as a supporting substrate 108. Here, a substrate other than a silicon substrate may be used. A silicon oxide film (the device isolation insulating film) 103 is then formed in an upper portion of the supporting substrate 108. This device isolation insulating film 103 is formed by burying a silicon oxide film in grooves formed in the supporting substrate 108, for example.

The silicon oxide film 110 is then formed by oxidizing an upper portion of the supporting substrate 108, for example. An n-type polycrystalline silicon film, for example, is then formed as a semiconductor film on the silicon oxide film 110. The n-type polycrystalline silicon film is formed by CVD or the like, while being doped with an n-type impurity, for example. Patterning is then performed on the semiconductor film, to form the semiconductor film 111FG and the semiconductor film 111CG. After that, the impurity is implanted into the supporting substrate 108, and activation annealing is performed, to form diffusion layers (the source/drain regions SD) on both sides of the floating gate electrode FG.

A phosphorus-doped or undoped polycrystalline silicon film, for example, is then formed as a semiconductor film on the silicon oxide film 110 as well as on the semiconductor film 111FG and the semiconductor film 111CG. In a case where undoped polycrystalline silicon is deposited, the n-type impurity is made to diffuse into the undoped polycrystalline silicon by the annealing performed later. Patterning is then performed on the semiconductor film, to form the semiconductor film 112FG and the semiconductor film 112CG. By this patterning, the floating gate electrode FG formed with the film stack of the semiconductor film 111FG and the semiconductor film 112FG placed thereon, and the control gate electrode CG formed with the film stack of the semiconductor film 111CG and the semiconductor film 112CG placed thereon are formed.

The insulating film Z is then formed on the silicon oxide film 110 as well as on the floating gate electrode FG and the control gate electrode CG.

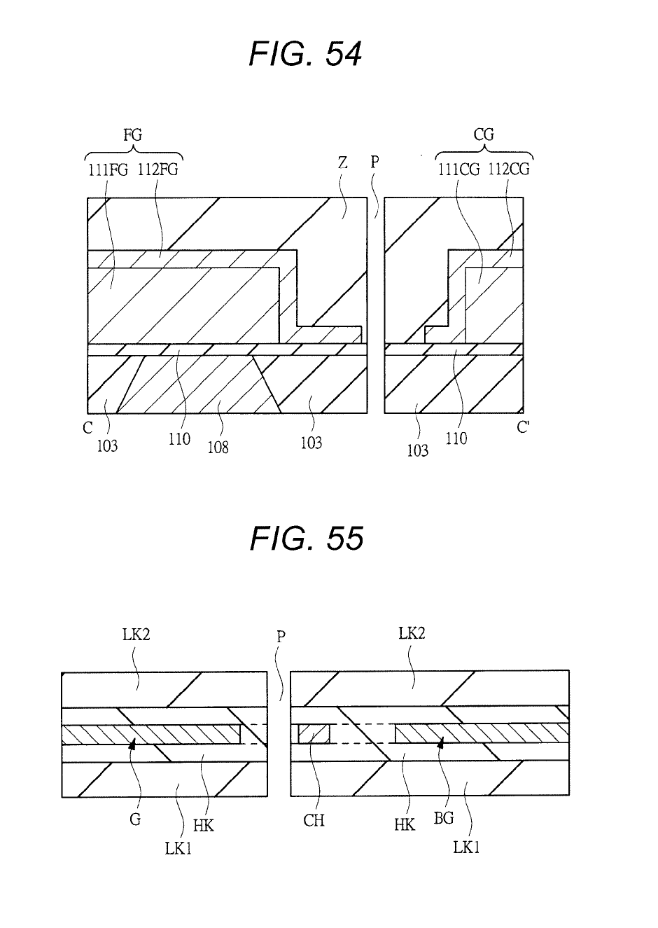

As shown in FIGS. 53 and 54, etching is performed on a lower portion of the supporting substrate 108, including the region in which the pore P is to be formed. In this manner, the thickness of the supporting substrate 108 is reduced to approximately 100 nm.

After that, the pore P penetrating through the insulating film Z, the silicon oxide film 110, and the device isolation insulating film 103 is formed between the floating gate electrode FG and the control gate electrode CG by irradiation of an energy beam such as a TEM beam.

Through the above described procedures, the semiconductor device of this embodiment is substantially completed. It should be noted that the above described procedures are merely examples, and the procedures that can be carried out are not limited to the above described ones.

### Fifth Embodiment

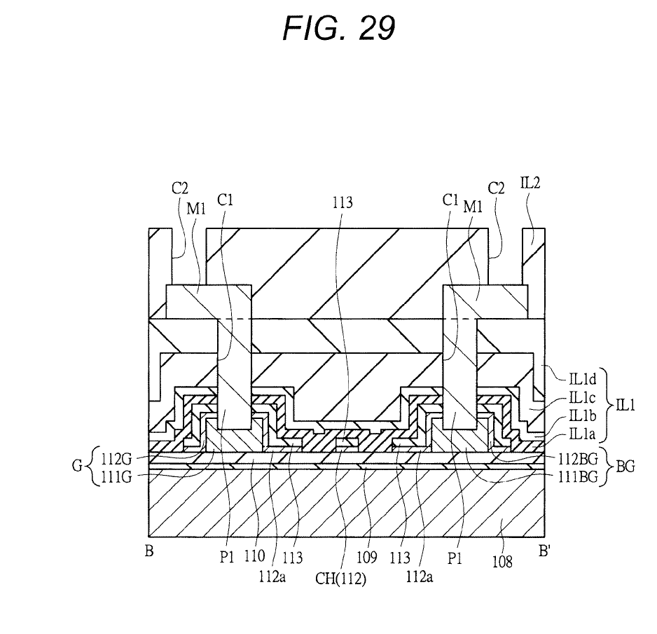

In the first embodiment (FIG. 29), the gate insulating film located between a side surface of the channel region CH and the gate electrode G is formed with the silicon oxide film IL1a. However, this gate insulating film may be formed with a high-k film.

FIG. 55 is a schematic cross-sectional view of the structure of a semiconductor device of this embodiment. As shown in FIG. 55, one side surface of a channel region CH and a side surface of a gate electrode G are arranged to face each other, and a pore P is located therebetween. The other side surface of the channel region CH and a side surface of a back gate electrode BG are arranged to face each other via the insulating film Z. A high-k film HK exists between the one side surface of the channel region CH and the side surface of the gate electrode G, and between the other side surface of the channel region CH and the side surface of the back gate electrode BG. A low-k film LK1 having a lower permittivity than that of the high-k film HK is placed under the high-k film HK, and another low-k film LK2 having a lower permittivity than that of the high-k film HK is placed on the high-k film HK.

As described above, the high-k film HK exists between the facing side surfaces of the channel region CH and the gate electrode G, and the permittivity thereof is made higher than that of the films (LK1 and LK2) located on and under the high-k film HK. With this arrangement, the electric field affecting the pore P does not easily scatter in the thickness direction of the gate electrode G (the vertical direction or the z-direction). Accordingly, the voltage applied to the gate electrode G is efficiently applied to the pore P. That is, an electric field is applied to the side surface of the channel region CH with high efficiency, and a large proportion of an inversion layer (the channel) can be formed in the side surface of the channel region CH. Accordingly, a test object can be examined with high sensitivity.

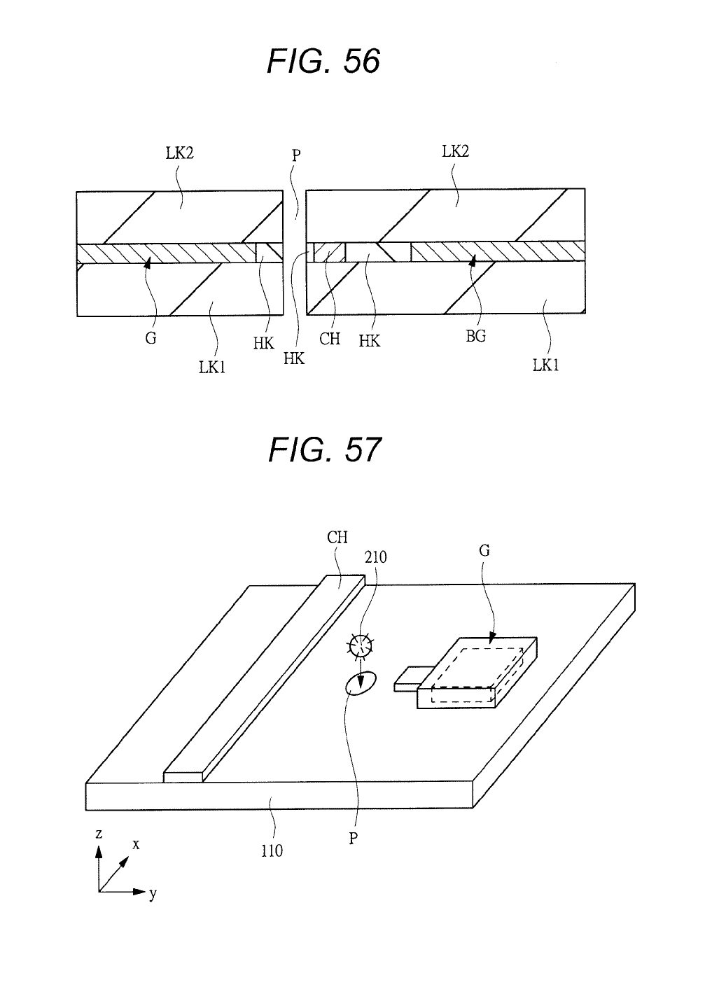

FIG. 56 is a schematic cross-sectional view of the structure of another semiconductor device of this embodiment. As shown in FIG. 56, the thickness of the portion of the high-k film HK located between the facing side surfaces of the channel region CH and the gate electrode G may be equal to the thickness of the channel region CH and the gate electrode G. Also, the thickness of the portion of the high-k film HK located between the facing side surfaces of the channel region CH and the back gate electrode BG is equal to the thickness of the channel region CH and the back gate electrode BG.

In this structure, it is even more difficult for the electric field affecting the pore P to scatter in the thickness direction of the gate electrode G. Accordingly, a test object can be examined with even higher sensitivity.

For example, the silicon oxide film IL1a of the first embodiment is replaced with the high-k film HK (a silicon nitride film, for example), and the silicon oxide film 110 and the silicon nitride film IL1b located under and on the silicon oxide film IL1a are replaced with low-k films (silicon oxide films, or LK1 and LK2, for example) having a lower permittivity than that of the silicon nitride film (see FIGS. 29 and others). With this arrangement, the above described effects can be achieved.

The structure of this embodiment can of course be applied not only to the semiconductor device of the first embodiment but to semiconductor devices of other embodiments (the second and third embodiments, and others). The structure of this embodiment can also be applied to the semiconductor device structure of the fifth embodiment described above. Specifically, the insulating film existing between the floating gate electrode FG and the control gate electrode CG is replaced with a film having a higher permittivity than those of the insulating films located on and under the high-k film. With this arrangement, an electric field can concentrate in the vicinity of the pore P, and influence of a test object in the pore P on the electric field can be clearly detected as changes in the source-drain current (the channel current).

### Sixth Embodiment

In the first embodiment, the DNA 200 as a test object is made to pass through the pore P, and a test (analysis) is then conducted (see FIGS. 2(A) and 2(B)). However, the test object is not limited to the form shown in FIGS. 2(A) and 2(B), and various modifications may be made. In this case, the radius (or the diameter) of the pore P is adjusted in accordance with sizes of test objects. FIG. 57 is a schematic perspective view of a semiconductor device of this embodiment.

As shown in FIG. 57, a bead 210 to which DNA adsorbs is used according to another DNA analysis method, for example. In this case, the diameter of the pore P is increased in accordance with the size of the bead 210 as the test object. For example, the diameter of the pore P is adjusted to approximately 100 nm.

In this case, hydrogen ion emission caused by reactions between the bead 210 having DNA adsorbing thereto and a reagent is detected. The amounts of hydrogen ion generation are regarded as changes in the source-drain current (the channel current), and accordingly, the nucleotide sequence of the DNA can be analyzed.

As described above, there are no restrictions on test objects, and not only test targets but also substances carrying test targets may be measured. Also, a test (analysis) may be conducted by regarding reaction products as changes in the source-drain current (the channel current) as described above.

In a case where reaction products are to be detected, the pore P is not necessarily a through hole, but may be a concavity (a concave portion). As reactions occur in the concavity (the concave portion), the reaction products should be detected.

The semiconductor device can be used not only for DNA analysis (nucleotide sequence analysis) but also for other purposes. For example, the semiconductor device can be used for testing whether specific nucleotides are included in single-strand DNA. In this case, a check can be made to determine whether a complementary base is hybridized to the specific nucleotides. At a site of hybridization, the charge amount is twice larger than that at the corresponding site of a single strand. For example, a specific base is added to unknown nucleotides, and the resultant substance serving as a test object is made to pass through the pore P of a semiconductor device described in one of the above described first through fifth embodiments and the like. In a case where the source-drain current (the channel current) decreases due to influence of a charge amount, for example, hybridization is confirmed, and it is clear that the specific nucleotides are included. In a case where the source-drain current (the channel current) does not change or the rate of change therein is low, the existence of the specific nucleotides is not confirmed.

### Seventh Embodiment

In this embodiment, the material forming the channel region CH and the material forming the pore P are described.

First, the material forming the channel region CH is described. Although a semiconductor film formed with an undoped polycrystalline silicon film or the like is used as the channel region CH in the first embodiment, other materials may be used.

For example, graphene or carbon nanotubes can be used as the material to form the channel region CH. The structure is the same as the structure of the first embodiment, except for the material forming the channel region CH (see FIG. 1 and others).

Graphene is one-atom-thick planar sheets of sp2-bonded carbon atoms. Graphene has a structure in which hexagonal lattices formed with carbon atoms and bonds thereof are two-dimensionally linked. Thus, graphene has an ideal two-dimensional atomic arrangement. Accordingly, a one-dimensional current path can be formed at the edge of the channel region CH facing the gate electrode G by forming the channel region CH from graphene and controlling the voltages to be applied to the gate electrode G and the back gate electrode BG. That is, an ideal thinnest current path in which monoelectrons are aligned in the x-direction can be formed.

A one-dimensional current path can also be formed at the edge of the channel region CH facing the gate electrode G by forming the channel region CH from carbon nanotubes and controlling the voltages to be applied to the gate electrode G and the back gate electrode BG.

As graphene or carbon nanotubes are used as the material to form the channel region CH in the above described manner, a minute field change due to a test object in the pore P can be sensed with high sensitivity. Accordingly, the rate of change in detection signals (detection sensitivity) can be made higher.

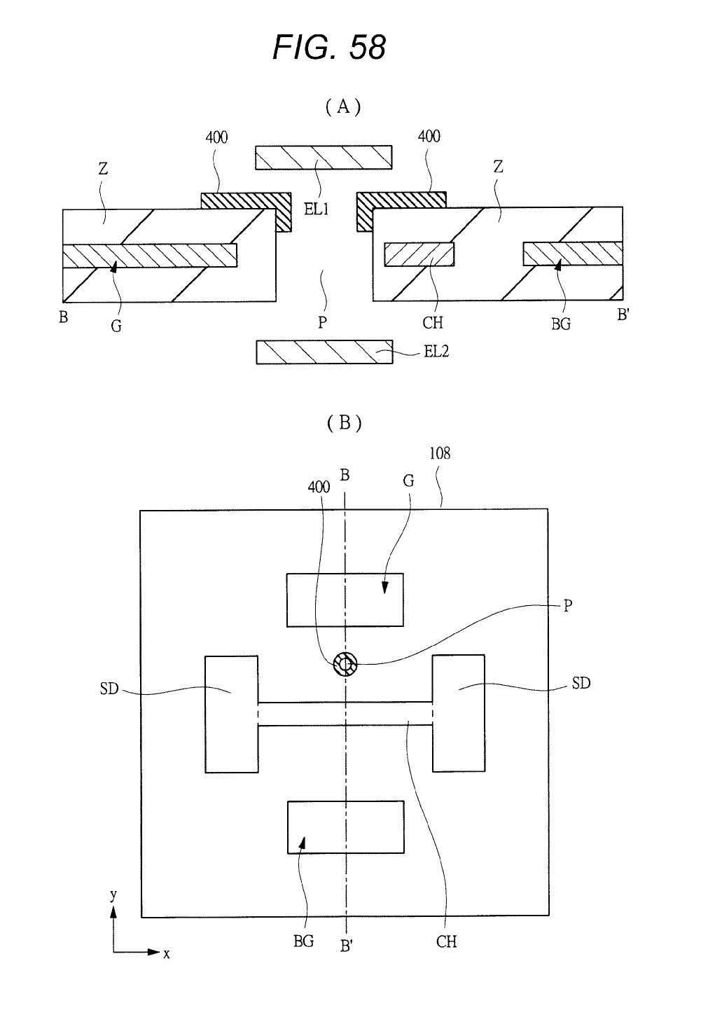

Next, the material forming the portion of the pore P is described. FIGS. 58(A) and 58(B) are a schematic cross-sectional view and a schematic plan view of the structure of the portion in the vicinity of the pore in a semiconductor device of this embodiment. FIG. 58(A) is the cross-sectional view, and FIG. 58(B) is the plan view. The cross-sectional view is equivalent to the B-B′ cross-section of the plan view.

As shown in FIG. 58(A), one side surface of the channel region CH and a side surface of the gate electrode G are arranged to face each other, and the pore P is located therebetween. This pore P is formed in the insulating film Z. The other side surface of the channel region CH and a side surface of the back gate electrode BG are arranged to face each other. Electrodes EL1 and EL2 are placed above and below the pore P. As shown in FIG. 58(B), the source/drain regions SD are placed at both ends of the channel region CH. In FIG. 58(B), the electrode EL1 and the electrode EL2 are not shown.

A biomembrane 400 formed with alpha hemolysin or the like is placed in the vicinity of the pore P. As shown in FIGS. 58(A) and 58(B), the biomembrane 400 is placed on an upper portion of the sidewall of the pore P and on the insulating film Z, so as to surround the pore P.

There are reports that, when DNA is made to pass through a nanopore formed with the biomembrane 400 of alpha hemolysin or the like, the value of the ion current passing through the nanopore changes with four types of nucleotides.

For example, a voltage difference is generated between the electrode EL1 and the electrode EL2 shown in FIG. 58(A), and DNA is made to pass through the pore P. At this point, the value of the ion current flowing between the electrode EL1 and the electrode EL2 varies among the respective nucleotides. That is, the ion density in the pore P varies among the respective nucleotides.

Therefore, the electric field to be applied to the channel region CH changes due to variations in the ion density in the pore P. Accordingly, variations in the ion density in the pore P can be measured as variations in the current between the source/drain regions SD (the channel current). In this manner, the respective nucleotides constituting DNA can be identified.

As minute changes in the charge amount in the pore P are detected as changes in the current of the FET, very large current changes can be detected. For example, the changes are much larger than variations in the value of the ion current flowing between the electrode EL1 and the electrode EL2, and accordingly, detection sensitivity can be increased.

### Eighth Embodiment

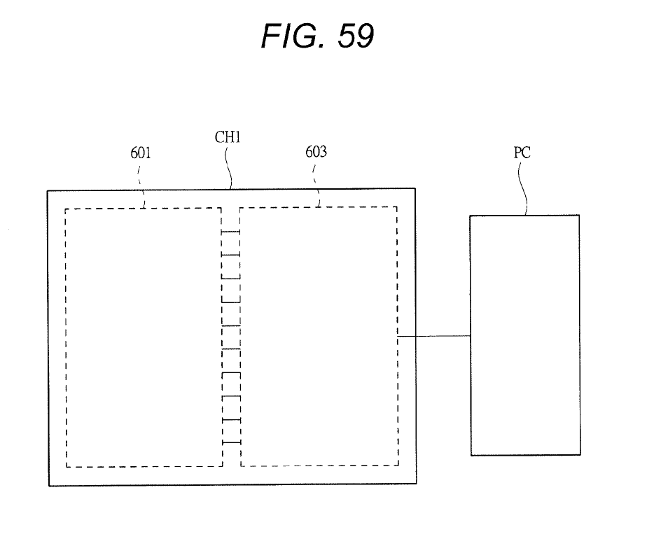

While there are no restrictions on applications of the semiconductor devices (semiconductor devices for detecting bio-related substances, for example) described above in the first through seventh embodiments, the semiconductor devices can be incorporated into systems for detecting bio-related substances as described below. FIGS. 59 through 63 are block diagrams schematically showing the configurations of systems according to this embodiment.

The system shown in FIG. 59 includes an array unit 601 and a signal processing circuit unit 603 on a semiconductor chip CH1.

In the array unit 601, single FETs (FET sensors) of any of the above described first through seventh embodiments are vertically and horizontally arranged in an array.

Using an ADC unit or the like, the signal processing circuit unit 603 converts signals detected by the respective FETs of the array unit 601, and performs signal processing.

The signals output from the signal processing circuit unit 603 are calculated by the computer PC, and are displayed as a sequence of four types of nucleotides.

As described above, the FETs of the above described first through seventh embodiments have structures that can be made smaller in size, and can be readily arranged in an array by using a semiconductor process. Accordingly, a smaller system can be realized, and costs can be lowered. Furthermore, as each nucleotide is detected as source-drain current (channel current), it is easy to perform conversion to signals and signal processing, and data can be collected in a suitable form for analysis using a computer. Accordingly, high-precision genomic analysis can be conducted at high speed.

Also, a specimen formed by amplifying a DNA into two or more DNAs is used in each of the FETs in an array, and the respective DNAs are analyzed in parallel. In this manner, the number of signals to be detected becomes larger, and reliability of analysis results can be increased.

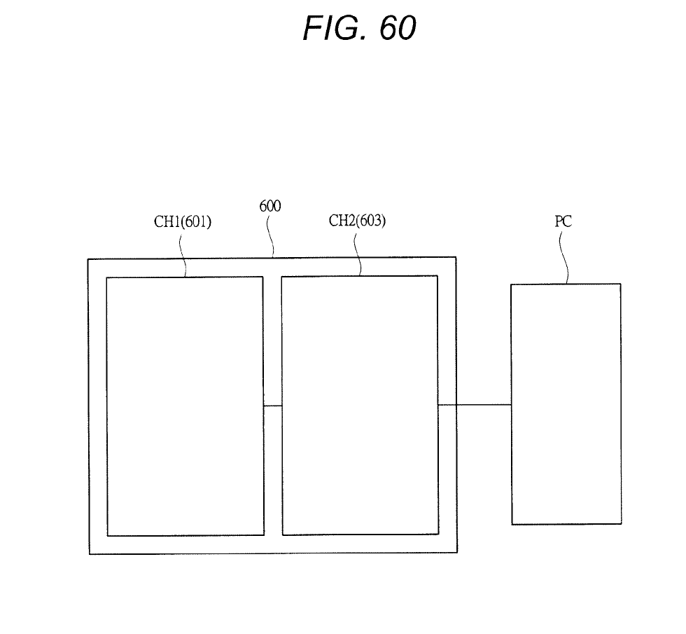

In the system shown in FIG. 60, an array unit 601 and a signal processing circuit unit 603 are placed on individual semiconductor chips CH1 and CH2, respectively. Those semiconductor chips (CH1 and CH2) are placed on a board (a mount board or a printed board) 600. The other aspects of the configuration are the same as those of FIG. 59.

As the array unit 601 is formed in an individual chip (CH1), the semiconductor chip CH1 (the array unit 601) that comes in contact with test objects can be easily replaced with another. For example, the semiconductor chip CH1 (the array unit 601) can be discarded every time a test is completed.

With this configuration, system costs can be lowered. Also, contamination of test objects can be prevented, and detection accuracy can be increased.

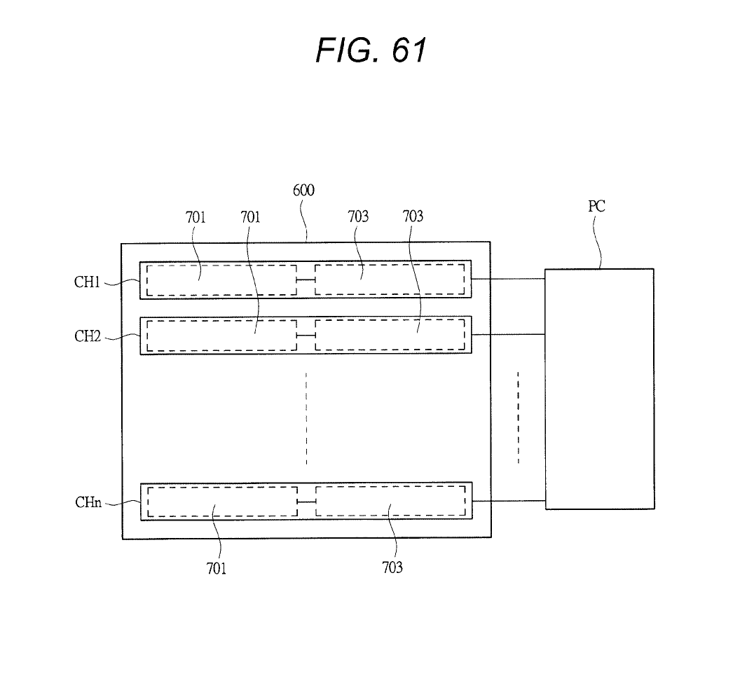

In the system shown in FIG. 61, an array unit 701 and a signal processing circuit unit 703 are divided into semiconductor chips CH1 through CHn. The semiconductor chips CH1 through CHn each include an array unit 701 and a signal processing circuit unit 703. Those semiconductor chips (CH1 through CHn) are placed on a board (a mount board or a printed board) 600. The other aspects of the configuration are the same as those of FIG. 60.

In the array units 701, single FETs (FET sensors) of any of the above described first through seventh embodiments are vertically and horizontally arranged in an array.

In this case, each array unit 701 is an array formed with a relatively small number of FETs. Each array unit 701 may be formed with a single FET.

As a semiconductor chip is divided into the semiconductor chips (CH1 through CHn) each including a relatively small number of FETs (an array unit 701), production load can be lowered. That is, decreases in yield due to FET defects can be restrained. Accordingly, the manufacturing of the system becomes easier, and the system yield becomes higher.

Also, the number of semiconductor chips (array units 701) mounted on the board 600 can be appropriately changed in accordance with the type of analysis. Accordingly, the number of FETs to be used for analysis can be easily adjusted, and system costs can be lowered.

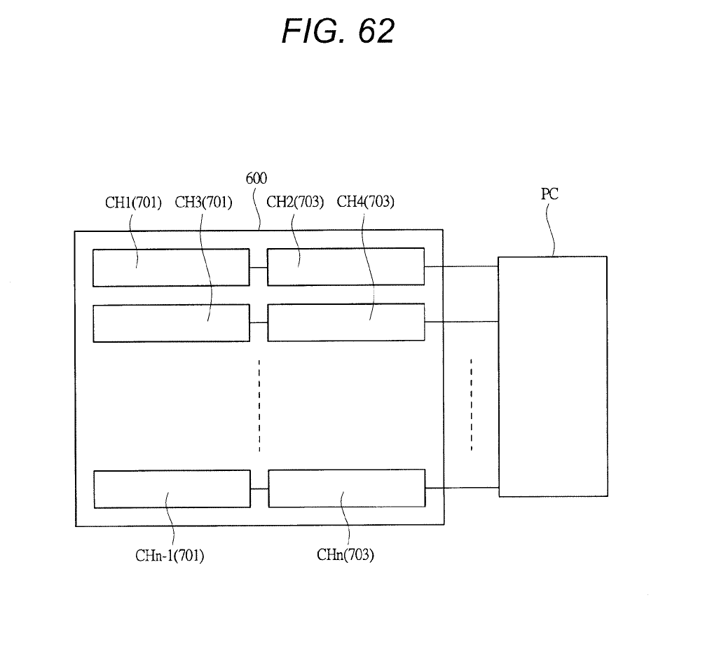

In the system shown in FIG. 62, array units 701 and signal processing circuit units 703 are placed on individual semiconductor chips CH1 through CHn, respectively. Those semiconductor chips (CH1 through CHn) are placed on a board (a mount board or a printed board) 600. In this case, the array unit 701 and the signal processing circuit unit 703 of each one semiconductor chip shown in FIG. 61 are formed in individual semiconductor chips.

In this case, a semiconductor chip is also divided into the semiconductor chips (CH1 through CHn) each including a relatively small number of FETs (an array unit 701). Accordingly, production load can be lowered. That is, decreases in yield due to FET defects or defects in the signal processing circuits can be restrained. Accordingly, the manufacturing of the system becomes easier, and the system yield becomes higher. Also, the number of semiconductor chips (array units 701) mounted on the board 600 can be appropriately changed in accordance with the type of analysis. Accordingly, the number of FETs to be used for analysis can be easily adjusted, and system costs can be lowered.

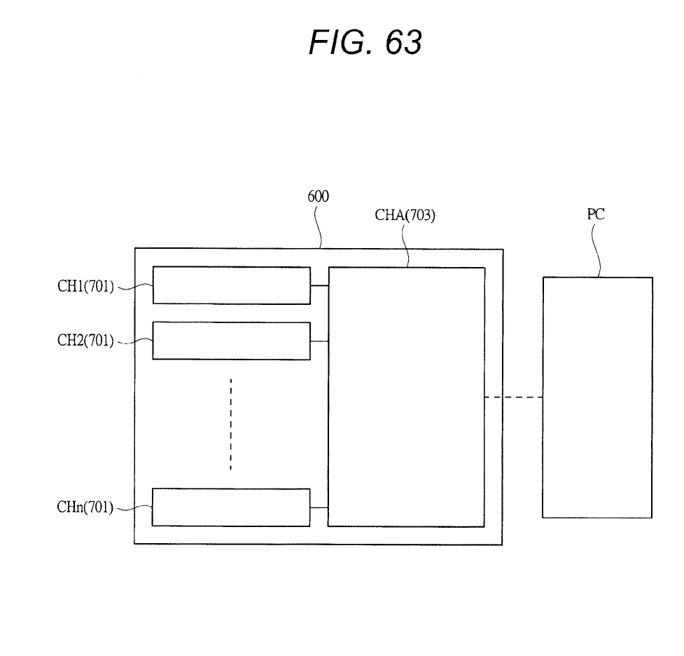

In the system shown in FIG. 63, array units 701 and a signal processing circuit unit 703 are placed on individual semiconductor chips (CH1 through CHn, and CHA), respectively. Those semiconductor chips (CH1 through CHn, and CHA) are placed on a board (a mount board or a printed board) 600. In this case, the signal processing circuit units 703 shown in FIG. 62 are placed on the single semiconductor chip CHA.

In this case, a semiconductor chip is also divided into the semiconductor chips (CH1 through CHn) each including a relatively small number of FETs (an array unit). Accordingly, production load can be lowered. That is, decreases in yield due to FET defects can be restrained. Accordingly, the manufacturing of the system becomes easier, and the system yield becomes higher. Also, the number of semiconductor chips (array units, or CH1 through CHn) mounted on the board 600 can be appropriately changed in accordance with the type of analysis. Accordingly, the number of FETs to be used for analysis can be easily adjusted, and system costs can be lowered.

Although the invention made by the inventor has been described in detail based on embodiments thereof, the present invention is not limited to the above embodiments, and various changes may of course be made to them without departing from the scope of the invention.

### INDUSTRIAL APPLICABILITY

The present invention relates to semiconductor devices, and can be appropriately applied to semiconductor devices particularly for detecting various kinds of substances including biological matter such as DNA.

### REFERENCE SIGNS LIST

10 inversion layer
103 device isolation insulating film
108 supporting substrate
109 silicon nitride film
110 silicon oxide film
111 semiconductor film
111BG semiconductor film
111CG semiconductor film
111FG semiconductor film
111G semiconductor film
112 semiconductor film
112BG semiconductor film
112CG semiconductor film
112FG semiconductor film
112G semiconductor film
112SD semiconductor film
112a protruding portion
113 silicon oxide film
117 hard mask
200 DNA
210 bead
400 biomembrane
600 board
601 array unit
603 signal processing circuit unit
701 array unit
703 signal processing circuit unit
BG back gate electrode
C1 contact hole
C2 contact hole
CG control gate electrode
CH channel region
CH1-CHn semiconductor chip
CHA semiconductor chip
Ca1 capacitance
Ca2 capacitance
DT Si dot
EL1, EL2 electrode
FG floating gate electrode
G gate electrode
GR groove
HK high-k film
IL1 interlayer insulating film
IL1a silicon oxide film
IL1b silicon nitride film
IL1c silicon oxide film
IL1d silicon nitride film
IL2 interlayer insulating film
LK1 low-k film
LK2 low-k film
M1 first interconnect layer
OA opening
P pore
P1 first plug
SD source/drain region
Z insulating film
xz1 side surface

1. A semiconductor device comprising:
a first semiconductor film placed on a first surface of an insulating layer;
source/drain regions placed on both sides of the first semiconductor film;
a gate electrode placed on the first surface, the gate electrode being at a distance from the first semiconductor film, the gate electrode being located to face a first side surface of the first semiconductor film;
a first insulating film located between the first semiconductor film and the gate electrode; and
a hole extending parallel to the first side surface of the first semiconductor film, the hole being perpendicular to the first surface.

2. The semiconductor device according to claim 1, wherein the hole is formed in the first insulating film.

3. The semiconductor device according to claim 1, wherein the hole is formed in a boundary portion between the first semiconductor film and the first insulating film.

4. The semiconductor device according to claim 1, wherein the hole is formed in the first semiconductor film.

5. The semiconductor device according to claim 1, wherein
a test object is introduced into the pore, and
the semiconductor device detects a field change caused by the test object in an inversion layer formed in the first side surface of the first semiconductor film, as a change in a current flowing between the source/drain regions.

6. The semiconductor device according to claim 1, further comprising
a back gate electrode placed on the first surface, the back gate electrode being at a distance from the first semiconductor film, the back gate electrode being located to face a second side surface of the first semiconductor film.

7. The semiconductor device according to claim 1, wherein
the gate electrode includes a second semiconductor film and a third semiconductor film located on the second semiconductor film, and
the third semiconductor film covers an upper portion of the second semiconductor film and a side surface of the second semiconductor film, and extends onto an upper surface of the insulating layer, the side surface of the second semiconductor film facing the first semiconductor film.

8. The semiconductor device according to claim 7, wherein a planar shape of an end portion of a section of the gate electrode is a triangular shape when seen from above, the section being formed on the insulating layer.

9. The semiconductor device according to claim 1, wherein the first insulating film is a film having higher permittivity than the insulating layer.

10. The semiconductor device according to claim 5, wherein the test object is a substance carrying a test target.

11. The semiconductor device according to claim 1, further comprising
a biomembrane placed near the hole.

12. The semiconductor device according to claim 1, wherein a diameter of the hole is not larger than 5 nm.

13. The semiconductor device according to claim 1, wherein a thickness of the first semiconductor film is not larger than 5 nm.

14. A semiconductor device comprising:
a first electrode placed on a first surface of an insulating layer;
a second electrode placed on the first surface, the second electrode being at a distance from the first electrode, the second electrode being located to face a first side surface of the first electrode;
a first insulating film located between the first electrode and the second electrode;
a hole formed in the first insulating film, the hole extending parallel to the first side surface of the first electrode, the hole being perpendicular to the first surface; and
source/drain electrodes facing a second surface of the insulating layer, the source/drain electrodes being located on both sides of the first electrode.

15. A method of manufacturing a semiconductor device, comprising:
(a) forming a first semiconductor film on a first surface of an insulating layer, and patterning the first semiconductor film to form a first film piece, a second film piece, and a third film piece;
(b) forming a second semiconductor film on the first film piece, the second film piece, and the third film piece;
(c) thinning the second semiconductor film by oxidizing a surface of the second semiconductor film;
(d) forming a semiconductor region from the second semiconductor film connecting the first film piece and the second film piece by patterning the second semiconductor film; and
(e) forming a hole in a region located between the semiconductor region and the third film piece, the region including an inside portion of the semiconductor region.
